(19) United States
(12) Patent Application Publication
(10) Pub. No.: US 2017/0275664 A1
(43) Pub. Date: Sep. 28, 2017

(54) Milling Process

(71) Applicant: Novozymes A/S, Bagsvaerd (DK)

(72) Inventors: Wang Han, Beijing (CN); Paria Saunders, Knightdale, NC (US); Scott R. McLaughlin, Wake Forest, NC (US); Zhen Long, Beijing (CN)

(73) Assignee: Novozymes A/S, Bagsvaerd (DK)

(21) Appl. No.: 15/616,108

(22) Filed: Jun. 7, 2017

Related U.S. Application Data
(63) Continuation of application No. 14/646,564, filed on May 21, 2015.
(60) Provisional application No. 61/748,949, filed on Jan. 4, 2013.

(30) Foreign Application Priority Data
Nov. 27, 2012 (CN) PCT/CN2012/085347

(51) Int. Cl. C12P 19/14; C12P 19/04; C08B 30/04; C12P 19/02

(52) U.S. Cl. CPC C12P 19/14; C12P 2201/00; C08B 30/044; C12P 19/02; C12P 19/04

(57) ABSTRACT

The present invention provides process for treating crop kernels, comprising the steps of a) soaking kernels in water to produce soaked kernels; b) grinding the soaked kernels; c) treating the soaked kernels in the presence of an effective amount of a beta-xylosidase, wherein step c) is performed before, during or after step b).

### CROSS-REFERENCE TO RELATED APPLICATIONS

This application is a continuation of U.S. application Ser. No. 14/646,564 filed on May 21, 2015 (pending), which is a 35 U.S.C. 371 national application of international application no. PCT/CN2013/087855 filed Nov. 26, 2013, which claims priority or the benefit under 35 U.S.C. 119 of Chinese PCT application no. PCT/CN2012/085347 filed Nov. 27, 2012 and U.S. provisional application No. 61/748,949 filed Jan. 4, 2013, the contents of which are fully incorporated herein by reference.

### REFERENCE TO SEQUENCE LISTING

This application contains a Sequence Listing in computer readable form. The computer readable form is incorporated herein by reference.

### FIELD OF THE INVENTION

The present invention relates to an improved process of treating crop kernels to provide a starch product of high quality suitable for conversion of starch into mono- and oligosaccharides, ethanol, sweeteners, etc. Further, the invention also relates to an enzyme composition comprising one or more enzyme activities suitable for the process of the invention and to the use of the composition of the invention.

### BACKGROUND OF THE INVENTION

Before starch, which is an important constituent in the kernels of most crops, such as corn, wheat, rice, sorghum bean, barley or fruit hulls, can be used for conversion of starch into saccharides, such as dextrose, fructose; alcohols, such as ethanol; and sweeteners, the starch must be made available and treated in a manner to provide a high purity starch. If starch contains more than 0.5% impurities, including the proteins, it is not suitable as starting material for starch conversion processes. To provide such pure and high quality starch product starting out from the kernels of crops, the kernels are often milled, as will be described further below.

Wet milling is often used for separating corn kernels into its four basic components: starch, germ, fiber and protein.

Typically wet milling processes comprise four basic steps. First the kernels are soaked or steeped for about 30 minutes to about 48 hours to begin breaking the starch and protein bonds. The next step in the process involves a coarse grind to break the pericarp and separate the germ from the rest of the kernel. The remaining slurry consisting of fiber, starch and protein is finely ground and screened to separate the fiber from the starch and protein. The starch is separated from the remaining slurry in hydrocyclones. The starch then can be converted to syrup or alcohol, or dried and sold as corn starch or chemically or physically modified to produce modified corn starch.

The use of enzymes has been suggested for the steeping step of wet milling processes. The commercial enzyme product Steepzyme® (available from Novozymes A/S) has been shown suitable for the first step in wet milling processes, i.e., the steeping step where corn kernels are soaked in water.

More recently, “enzymatic milling”, a modified wet-milling process that uses proteases to significantly reduce the total processing time during corn wet milling and eliminates the need for sulfur dioxide as a processing agent, has been developed. Johnston et al., Cereal Chem, 81, p. 626-632 (2004).

U.S. Pat. No. 6,566,125 discloses a method for obtaining starch from maize involving soaking maize kernels in water to produce soaked maize kernels, grinding the soaked maize kernels to produce a ground maize slurry, and incubating the ground maize slurry with enzyme (e.g., protease).

U.S. Pat. No. 5,066,218 discloses a method of milling grain, especially corn, comprising cleaning the grain, steeping the grain in water to soften it, and then milling the grain with a cellulase enzyme.

WO 2002/000731 discloses a process of treating crop kernels, comprising soaking the kernels in water for 1-12 hours, wet milling the soaked kernels and treating the kernels with one or more enzymes including an acidic protease.

WO 2002/000911 discloses a process of starch gluten separation, comprising subjecting mill starch to an acidic protease.

WO 2002/002644 discloses a process of washing a starch slurry obtained from the starch gluten separation step of a milling process, comprising washing the starch slurry with an aqueous solution comprising an effective amount of acidic protease.

There remains a need for improvement of processes for providing starch suitable for conversion into mono- and oligo-saccharides, ethanol, sweeteners, etc.

### SUMMARY OF THE INVENTION

The invention provides a process for treating crop kernels, comprising the steps of a) soaking kernels in water to produce soaked kernels; b) grinding the soaked kernels; c) treating the soaked kernels in the presence of a beta-xylosidase, wherein step c) is performed before, during or after step b).

In one embodiment, the invention provides the use of a beta-xylosidase to enhance the wet milling benefit of one or more enzymes.

### DETAILED DESCRIPTION OF THE INVENTION

Accordingly, it is an object of the invention to provide improved processes of treating crop kernels to provide starch of high quality.

In one embodiment, the enzyme compositions useful in the processes of the invention provide benefits including, improving starch yield and/or purity, improving gluten quality and/or yield, improving fiber, gluten, or steep water filtration, dewatering and evaporation, easier germ separation and/or better post-saccharification filtration, and process energy savings thereof.

Without wishing to be bound by theory, the present inventors have discovered the use of beta-xylosidase in wet milling and in particular, the use of beta-xylosidase in combination with xylanase, can provide a significant increase in, e.g., starch and gluten yields. The use of beta-xylosidase is believed to be specifically important in the liberation of additional starch and gluten from the fiber fraction, thus facilitating yield increases.

This can provide a benefit to the industry, e.g., on the basis of cost and ease of use.

Definitions of Enzymes

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. For purposes of the present invention, one unit of beta-xylosidase is defined as 1.0 μmole of p-nitrophenolate anion produced per minute at 40° C., pH 5 from 1 mM p-nitrophenyl-beta-D-xyloside as substrate in 100 mM sodium citrate containing 0.01% TWEEN® 20.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teeri, 1997, Crystalline cellulose degradation: New insight into the function of cellobiohydrolases, Trends in Biotechnology 15: 160-167; Teeri et al., 1998, Trichoderma reesei cellobiohydrolases: why so efficient on crystalline cellulose?, Biochem. Soc. Trans. 26: 173-178). Cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581. In the present invention, the Tomme et al. method can be used to determine cellobiohydrolase activity.

Cellulolytic enzyme composition, cellulase or cellulase preparation: The term “cellulolytic enzyme composition, “cellulase” or cellulase preparation means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

Cellulosic material: The term “cellulosic material” means any material containing cellulose. Cellulose is a homopolymer of anyhdrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Hemicellulolytic enzyme or hemicellulase: The term “hemicellulolytic enzyme” or “hemicellulase” means one or more (e.g., several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) data-base. Hemicellulolytic enzyme activities can be measured according to Ghose and Bisaria, 1987, Pure & Appl. Chem. 59: 1739-1752, at a suitable temperature, e.g., 50° C., 55° C., or 60° C., and pH, e.g., 5.0 or 5.5.

Polypeptide having cellulolytic enhancing activity: The term “polypeptide having cellulolytic enhancing activity” means a GH61 polypeptide that catalyzes the enhancement of the hydrolysis of a cellulosic material by enzyme having cellulolytic activity. In one aspect, a mixture of CELLUCLAST® 1.5 L (Novozymes A/S, Bagsvrd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The GH61 polypeptides having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Protease: The term “proteolytic enzyme” or “protease” means one or more (e.g., several) enzymes that break down the amide bond of a protein by hydrolysis of the peptide bonds that link amino acids together in a polypeptide chain.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, Recent progress in the assays of xylanolytic enzymes, 2006, Journal of the Science of Food and Agriculture 86(11): 1636-1647; Spanikova and Biely, 2006, Glucuronoyl esterase—Novel carbohydrate esterase produced by Schizophyllum commune, FEBS Letters 580(19): 4597-4601; Herrmann, Vrsanska, Jurickova, Hirsch, Biely, and Kubicek, 1997, The beta-D-xylosidase of Trichoderma reesei is a multifunctional beta-D-xylan xylohydrolase, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. The most common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 (4-(1,1,3,3-tetramethylbutyl)phenyl-polyethylene glycol) and 200 mM sodium phosphate buffer pH 6 at 37° C. One unit of xylanase activity is defined as 1.0 micromole of azurine produced per minute at 37° C., pH 6 from 0.2% AZCL-arabinoxylan as substrate in 200 mM sodium phosphate pH 6 buffer.

For purposes of the present invention, xylan degrading activity is determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, A new reaction for colorimetric determination of carbohydrates, Anal. Biochem 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate buffer pH 6 at 37° C. One unit of xylanase activity is defined as 1.0 micromole of azurine produced per minute at 37° C., pH 6 from 0.2% AZCL-arabinoxylan as substrate in 200 mM sodium phosphate pH 6 buffer.

Other Definitions

Crop kernels: The term “crop kernels” includes kernels from, e.g., corn (maize), rice, barley, sorghum bean, fruit hulls, and wheat. Corn kernels are exemplary. A variety of corn kernels are known, including, e.g., dent corn, flint corn, pod corn, striped maize, sweet corn, waxy corn and the like.

In an embodiment, the corn kernel is yellow dent corn kernel. Yellow dent corn kernel has an outer covering referred to as the “Pericarp” that protects the germ in the kernels. It resists water and water vapour and is undesirable to insects and microorganisms.

The only area of the kernels not covered by the “Pericarp” is the “Tip Cap”, which is the attachment point of the kernel to the cob.

Germ: The “Germ” is the only living part of the corn kernel. It contains the essential genetic information, enzymes, vitamins, and minerals for the kernel to grow into a corn plant. In yellow dent corn, about 25 percent of the germ is corn oil. The endosperm covered surrounded by the germ comprises about 82 percent of the kernel dry weight and is the source of energy (starch) and protein for the germinating seed. There are two types of endosperm, soft and hard. In the hard endosperm, starch is packed tightly together. In the soft endosperm, the starch is loose.

Starch: The term “starch” means any material comprised of complex polysaccharides of plants, composed of glucose units that occurs widely in plant tissues in the form of storage granules, consisting of amylose and amylopectin, and represented as (C6H1005)n, where n is any number.

Milled: The term “milled” refers to plant material which has been broken down into smaller particles, e.g., by crushing, fractionating, grinding, pulverizing, etc.

Grind or grinding: The term “grinding” means any process that breaks the pericarp and opens the crop kernel.

Steep or steeping: The term “steeping” means soaking the crop kernel with water and optionally SO2.

Dry solids: The term “dry solids” is the total solids of a slurry in percent on a dry weight basis.

Oligosaccharide: The term “oligosaccharide” is a compound having 2 to 10 monosaccharide units.

Wet milling benefit: The term “wet milling benefit” means one or more of improved starch yield and/or purity, improved gluten quality and/or yield, improved fiber, gluten, or steep water filtration, dewatering and evaporation, easier germ separation and/or better post-saccharification filtration, and process energy savings thereof.

Allelic variant: The term “allelic variant” means any of two or more (e.g., several) alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Fragment: The term “fragment” means a polypeptide having one or more (e.g., several) amino acids absent from the amino and/or carboxyl terminus of a mature polypeptide main; wherein the fragment has enzyme activity. In one aspect, a fragment contains at least 85%, e.g., at least 90% or at least 95% of the amino acid residues of the mature polypeptide of an enzyme.

High stringency conditions: The term “high stringency conditions” means for probes of at least 100 nucleotides in length, prehybridization and hybridization at 42° C. in 5× SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Low stringency conditions: The term “low stringency conditions” means for probes of at least 100 nucleotides in length, prehybridization and hybridization at 42° C. in 5× SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Mature polypeptide: The term “mature polypeptide” means a polypeptide in its final form following translation and any post-translational modifications, such as N terminal processing, C terminal truncation, glycosylation, phosphorylation, etc.

It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having enzyme activity.

Medium stringency conditions: The term “medium stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5× SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Parent Enzyme: The term “parent” means an enzyme to which an alteration is made to produce a variant. The parent may be a naturally occurring (wild-type) polypeptide or a variant thereof.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having enzyme activity. In one aspect, a subsequence contains at least 85%, e.g., at least 90% or at least 95% of the nucleotides of the mature polypeptide coding sequence of an enzyme.

Variant: The term “variant” means a polypeptide having enzyme or enzyme enhancing activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

In one aspect, the variant differs by up to 10 amino acids, e.g., 1, 2, 3, 4, 5, 6, 7, 8, 9, or 10, from the mature polypeptide of a SEQ ID NO: as identified herein. In another embodiment, the present invention relates to variants of the mature polypeptide of a SEQ ID NO: herein comprising a substitution, deletion, and/or insertion at one or more (e.g., several) positions. In an embodiment, the number of amino acid substitutions, deletions and/or insertions introduced into the mature polypeptide of a SEQ ID NO: herein is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function Wild-Type Enzyme: The term “wild-type” enzyme means an enzyme expressed by a naturally occurring microorganism, such as a bacterium, yeast, or filamentous fungus found in nature.

The Milling Process

The kernels are milled in order to open up the structure and to allow further processing and to separate the kernels into the four main constituents: starch, germ, fiber and protein.

In one embodiment, a wet milling process is used. Wet milling gives a very good separation of germ and meal (starch granules and protein) and is often applied at locations where there is a parallel production of syrups.

The inventors of the present invention have surprisingly found that the quality of the starch final product may be improved by treating crop kernels in the processes as described herein.

The processes of the invention result in comparison to traditional processes in a higher starch quality, in that the final starch product is more pure and/or a higher yield is obtained and/or less process time is used. Another advantage may be that the amount of chemicals, such as SO2 and NaHSO3, which need to be used, may be reduced or even fully removed.

Wet Milling

Starch is formed within plant cells as tiny granules insoluble in water. When put in cold water, the starch granules may absorb a small amount of the liquid and swell. At temperatures up to about 50° C. to 75° C. the swelling may be reversible. However, with higher temperatures an irreversible swelling called “gelatinization” begins. Granular starch to be processed according to the present invention may be a crude starch-containing material comprising (e.g., milled) whole grains including non-starch fractions such as germ residues and fibers. The raw material, such as whole grains, may be reduced in particle size, e.g., by wet milling, in order to open up the structure and allowing for further processing. Wet milling gives a good separation of germ and meal (starch granules and protein) and is often applied at locations where the starch hydrolyzate is used in the production of, e.g., syrups.

In an embodiment the particle size is reduced to between 0.05-3.0 mm, preferably 0.1-0.5 mm, or so that at least 30%, preferably at least 50%, more preferably at least 70%, even more preferably at least 90% of the starch-containing material fits through a sieve with a 0.05-3.0 mm screen, preferably 0.1-0.5 mm screen.

More particularly, degradation of the kernels of corn and other crop kernels into starch suitable for conversion of starch into mono- and oligo-saccharides, ethanol, sweeteners, etc. consists essentially of four steps:

1. Steeping and germ separation,

2. Fiber washing and drying,

3. Starch gluten separation, and

4. Starch washing.

1. Steeping and Germ Separation

Corn kernels are softened by soaking in water for between about 30 minutes to about 48 hours, preferably 30 minutes to about 15 hours, such as about 1 hour to about 6 hours at a temperature of about 50° C., such as between about 45° C. to 60° C. During steeping, the kernels absorb water, increasing their moisture levels from 15 percent to 45 percent and more than doubling in size. The optional addition of e.g. 0.1 percent sulfur dioxide (SO2) and/or NaHSO3 to the water prevents excessive bacteria growth in the warm environment. As the corn swells and softens, the mild acidity of the steepwater begins to loosen the gluten bonds within the corn and release the starch. After the corn kernels are steeped they are cracked open to release the germ. The germ contains the valuable corn oil. The germ is separated from the heavier density mixture of starch, hulls and fiber essentially by “floating” the germ segment free of the other substances under closely controlled conditions. This method serves to eliminate any adverse effect of traces of corn oil in later processing steps.

In an embodiment of the invention the kernels are soaked in water for 2-10 hours, preferably about 3-5 hours at a temperature in the range between 40 and 60° C., preferably around 50° C.

In one embodiment, 0.01-1%, preferably 0.05-0.3%, especially 0.1% SO2 and/or NaHSO3 may be added during soaking.

2. Fiber Washing and Drying

To get maximum starch recovery, while keeping any fiber in the final product to an absolute minimum, it is necessary to wash the free starch from the fiber during processing. The fiber is collected, slurried and screened to reclaim any residual starch or protein.

3. Starch Gluten Separation

The starch-gluten suspension from the fiber-washing step, called mill starch, is separated into starch and gluten. Gluten has a low density compared to starch. By passing mill starch through a centrifuge, the gluten is readily spun out.

4. Starch Washing.

The starch slurry from the starch separation step contains some insoluble protein and much of solubles. They have to be removed before a top quality starch (high purity starch) can be made. The starch, with just one or two percent protein remaining, is diluted, washed 8 to 14 times, rediluted and washed again in hydroclones to remove the last trace of protein and produce high quality starch, typically more than 99.5% pure.

Products

Wet milling can be used to produce, without limitation, corn steep liquor, corn gluten feed, germ, corn oil, corn gluten meal, cornstarch, modified corn starch, syrups such as corn syrup, and corn ethanol.

Enzymes

The enzyme(s) and polypeptides described below are to be used in an “effective amount” in processes of the present invention. Below should be read in context of the enzyme disclosure in the “Definitions”-section above.

Beta-Xylosidase

Examples of beta-xylosidases useful in the processes of the present invention include, but are not limited to, beta-xylosidases from Neurospora crassa (SwissProt accession number Q7SOW4), Trichoderma reesei (UniProtKB/TrEMBL accession number Q92458), and Talaromyces emersonii (SwissProt accession number Q8X212).

In one embodiment the beta-xylosidase is derived from the genus Aspergillus, such as a strain of Aspergillus fumigatus, such as the one described in WO 2011/057140 as SEQ ID NO: 206; or SEQ ID NO: 6 herein, or a beta-xylosidase having at least 80%, such as at least 85%, such such as at least 90%, preferably 95%, such as at least 96%, such as 97%, such as at least 98%, such as at least 99% identity to SEQ ID NO: 206 in WO 2011/057140 or SEQ ID NO: 6 herein. In one aspect, the beta-xylosidase differs by up to 10 amino acids, e.g., 1, 2, 3, 4, 5, 6, 7, 8, 9, or 10, from the mature polypeptide of SEQ ID NO: 6. In another embodiment, the present invention relates to variants of the mature polypeptide of SEQ ID NO: 6 comprising a substitution, deletion, and/or insertion at one or more (e.g., several) positions. In an embodiment, the number of amino acid substitutions, deletions and/or insertions introduced into the mature polypeptide of SEQ ID NO: 6 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

In one embodiment the beta-xylosidase is derived from a strain of the genus Aspergillus, such as a strain of Aspergillus fumigatus, such as the one disclosed in U.S. provisional No. 61/526,833 or PCT/US12/052163 or SEQ ID NO: 16 in WO 2013/028928 (Examples 16 and 17), or derived from a strain of Trichoderma, such as a strain of Trichoderma reesei, such as the mature polypeptide of SEQ ID NO: 58 in WO 2011/057140, or a beta-xylosidase having at least 80%, such as at least 85%, such such as at least 90%, preferably 95%, such as at least 96%, such as 97%, such as at least 98%, such as at least 99% identity thereto.

Additional Enzymes

Proteases

The protease may be any protease. Suitable proteases include microbial proteases, such as fungal and bacterial proteases. Preferred proteases are acidic proteases, i.e., proteases characterized by the ability to hydrolyze proteins under acidic conditions below pH 7. Preferred proteases are acidic endoproteases. An acid fungal protease is preferred, but also other proteases can be used.

The acid fungal protease may be derived from Aspergillus, Candida, Coriolus, Endothia, Enthomophtra, Irpex, Mucor, Penicillium, Rhizopus, Sclerotium, and Torulopsis. In particular, the protease may be derived from Aspergillus aculeatus (WO 95/02044), Aspergillus awamori (Hayashida et al., 1977, Agric. Biol. Chem. 42(5), 927-933), Aspergillus niger (see, e.g., Koaze et al., 1964, Agr. Biol. Chem. Japan 28: 216), Aspergillus saitoi (see, e.g., Yoshida, 1954, J. Agr. Chem. Soc. Japan 28: 66), or Aspergillus oryzae, such as the pepA protease; and acidic proteases from Mucor miehei or Mucor pusillus.

In an embodiment the acidic protease is a protease complex from A. oryzae sold under the tradename Flavourzyme® (from Novozymes A/S) or an aspartic protease from Rhizomucor miehei or Spezyme® FAN or GC 106 from Genencor Int.

In a preferred embodiment the acidic protease is an aspartic protease, such as an aspartic protease derived from a strain of Aspergillus, in particular A. aculeatus, especially A. aculeatus CBD 101.43.

Preferred acidic proteases are aspartic proteases, which retain activity in the presence of an inhibitor selected from the group consisting of pepstatin, Pefabloc, PMSF, or EDTA. Protease I derived from A. aculeatus CBS 101.43 is such an acidic protease.

In a preferred embodiment the process of the invention is carried out in the presence of the acidic Protease I derived from A. aculeatus CBS 101.43 in an effective amount.

In another embodiment the protease is derived from a strain of the genus Aspergillus, such as a strain of Aspergillus aculaetus, such as Aspergillus aculeatus CBS 101.43, such as the one disclosed in WO 95/02044, or a protease having at least 80%, such as at least 85%, such as at least 90%, preferably 95%, such as at least 96%, such as 97%, such as at least 98%, such as at least 99% identity protease of WO 95/02044. In one aspect, the protease differs by up to 10 amino acids, e.g., 1, 2, 3, 4, 5, 6, 7, 8, 9, or 10, from the mature polypeptide of WO 95/02044. In another embodiment, the present invention relates to variants of the mature polypeptide of WO 95/02044 comprising a substitution, deletion, and/or insertion at one or more (e.g., several) positions. In an embodiment, the number of amino acid substitutions, deletions and/or insertions introduced into the mature polypeptide of WO 95/02044 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

The protease may be a neutral or alkaline protease, such as a protease derived from a strain of Bacillus. A particular protease is derived from Bacillus amyloliquefaciens and has the sequence obtainable at Swissprot as Accession No. P06832. The proteases may have at least 90% sequence identity to the amino acid sequence disclosed in the Swissprot Database, Accession No. P06832 such as at least 92%, at least 95%, at least 96%, at least 97%, at least 98%, or particularly at least 99% identity.

The protease may have at least 90% sequence identity to the amino acid sequence disclosed as sequence 1 in WO 2003/048353 such as at 92%, at least 95%, at least 96%, at least 97%, at least 98%, or particularly at least 99% identity.

The protease may be a papain-like protease selected from the group consisting of proteases within EC 3.4.22.* (cysteine protease), such as EC 3.4.22.2 (papain), EC 3.4.22.6 (chymopapain), EC 3.4.22.7 (asclepain), EC 3.4.22.14 (actinidain), EC 3.4.22.15 (cathepsin L), EC 3.4.22.25 (glycyl endopeptidase) and EC 3.4.22.30 (caricain).

In an embodiment, the protease is a protease preparation derived from a strain of Aspergillus, such as Aspergillus oryzae. In another embodiment the protease is derived from a strain of Rhizomucor, preferably Rhizomucor miehei. In another embodiment the protease is a protease preparation, preferably a mixture of a proteolytic preparation derived from a strain of Aspergillus, such as Aspergillus oryzae, and a protease derived from a strain of Rhizomucor, preferably Rhizomucor miehei.

Aspartic acid proteases are described in, for example, Handbook of Proteolytic Enzymes, Edited by A. J. Barrett, N. D. Rawlings and J. F. Woessner, Academic Press, San Diego, 1998, Chapter 270. Examples of aspartic acid proteases include, e.g., those disclosed in Berka et al., 1990, Gene 96: 313; Berka et al., 1993, Gene 125: 195-198; and Gomi et al., 1993, Biosci. Biotech. Biochem. 57: 1095-1100, which are hereby incorporated by reference.

The protease also may be a metalloprotease, which is defined as a protease selected from the group consisting of:

(a) proteases belonging to EC 3.4.24 (metalloendopeptidases); preferably EC 3.4.24.39 (acid metallo proteinases);

(b) metalloproteases belonging to the M group of the above Handbook;

(c) metalloproteases not yet assigned to clans (designation: Clan MX), or belonging to either one of clans MA, MB, MC, MD, ME, MF, MG, MH (as defined at pp. 989-991 of the above Handbook);

(d) other families of metalloproteases (as defined at pp. 1448-1452 of the above Handbook);

(e) metalloproteases with a HEXXH motif;

(f) metalloproteases with an HEFTH motif;

(g) metalloproteases belonging to either one of families M3, M26, M27, M32, M34, M35, M36, M41, M43, or M47 (as defined at pp. 1448-1452 of the above Handbook);

(h) metalloproteases belonging to the M28E family; and

(i) metalloproteases belonging to family M35 (as defined at pp. 1492-1495 of the above Handbook).

In other particular embodiments, metalloproteases are hydrolases in which the nucleophilic attack on a peptide bond is mediated by a water molecule, which is activated by a divalent metal cation. Examples of divalent cations are zinc, cobalt or manganese. The metal ion may be held in place by amino acid ligands. The number of ligands may be five, four, three, two, one or zero. In a particular embodiment the number is two or three, preferably three.

There are no limitations on the origin of the metalloprotease used in a process of the invention. In an embodiment the metalloprotease is classified as EC 3.4.24, preferably EC 3.4.24.39. In one embodiment, the metalloprotease is an acid-stable metalloprotease, e.g., a fungal acidstable metalloprotease, such as a metalloprotease derived from a strain of the genus Thermoascus, preferably a strain of Thermoascus aurantiacus, especially Thermoascus aurantiacus CGMCC No. 0670 (classified as EC 3.4.24.39). In another embodiment, the metalloprotease is derived from a strain of the genus Aspergillus, preferably a strain of Aspergillus oryzae.

In one embodiment the metalloprotease has a degree of sequence identity to amino acids 159 to 177, or preferably amino acids 1 to 177 (the mature polypeptide) of SEQ ID NO: 1 of WO 2010/008841 (a Thermoascus aurantiacus metalloprotease) of at least 80%, at least 82%, at least 85%, at least 90%, at least 95%, or at least 97%; and which have metalloprotease activity.

The Thermoascus aurantiacus metalloprotease is a preferred example of a metalloprotease suitable for use in a process of the invention. Another metalloprotease is derived from Aspergillus oryzae and comprises SEQ ID NO: 11 disclosed in WO 2003/048353, or amino acids 23-353; 23-374; 23-397; 1-353; 1-374; 1-397; 177-353; 177-374; or 177-397 thereof, and SEQ ID NO: 10 disclosed in WO 2003/048353.

Another metalloprotease suitable for use in a process of the invention is the Aspergillus oryzae metalloprotease comprising SEQ ID NO: 5 of WO 2010/008841, or a metalloprotease is an isolated polypeptide which has a degree of identity to SEQ ID NO: 5 5 of at least about 80%, at least 82%, at least 85%, at least 90%, at least 95%, or at least 97%; and which have metalloprotease activity. In particular embodiments, the metalloprotease consists of the amino acid sequence of SEQ ID NO: 5.

In a particular embodiment, a metalloprotease has an amino acid sequence that differs by forty, thirty-five, thirty, twenty-five, twenty, or by fifteen amino acids from amino acids 159 to 177, or +1 to 177 of the amino acid sequences of the Thermoascus aurantiacus or Aspergillus oryzae metalloprotease.

In another embodiment, a metalloprotease has an amino acid sequence that differs by ten, or by nine, or by eight, or by seven, or by six, or by five amino acids from amino acids 159 to 177, or +1 to 177 of the amino acid sequences of these metalloproteases, e.g., by four, by three, by two, or by one amino acid.

In particular embodiments, the metalloprotease a) comprises or b) consists of i) the amino acid sequence of amino acids 159 to 177, or +1 to 177 of SEQ ID NO: 1 of WO 2010/008841;

ii) the amino acid sequence of amino acids 23-353, 23-374, 23-397, 1-353, 1-374, 1-397, 177-353, 177-374, or 177-397 of SEQ ID NO: 3 3 of WO 2010/008841;

iii) the amino acid sequence of SEQ ID NO: 5 of WO 2010/008841; or allelic variants, or fragments, of the sequences of i), ii), and iii) that have protease activity.

A fragment of amino acids 159 to 177, or +1 to 177 of SEQ ID NO: 1 of WO 2010/008841 or of amino acids 23-353, 23-374, 23-397, 1-353, 1-374, 1-397, 177-353, 177-374, or 177-397 of SEQ ID NO: 3 of WO 2010/008841; is a polypeptide having one or more amino acids deleted from the amino and/or carboxyl terminus of these amino acid sequences. In one embodiment a fragment contains at least 75 amino acid residues, or at least 100 amino acid residues, or at least 125 amino acid residues, or at least 150 amino acid residues, or at least 160 amino acid residues, or at least 165 amino acid residues, or at least 170 amino acid residues, or at least 175 amino acid residues.

In another embodiment, the metalloprotease is combined with another protease, such as a fungal protease, preferably an acid fungal protease.

In a preferred embodiment the protease is S53 protease 3 from Meripilus giganteus, e.g., the one disclosed in Examples 1 and 2 in PCT/EP2013/068361 (hereby incorporated by reference).

Commercially available products include ALCALASE®, ESPERASE™, FLAVOURZYME™, NEUTRASE®, RENNILASE®, NOVOZYM™ FM 2.0 L, and iZyme BA (available from Novozymes A/S, Denmark) and GC106™ and SPEZYME™ FAN from Genencor International, Inc., USA.

The protease may be present in an amount of 0.0001-1 mg enzyme protein per g dry solids (DS) kernels, preferably 0.001 to 0.1 mg enzyme protein per g DS kernels.

In an embodiment, the protease is an acidic protease added in an amount of 1-20,000 HUT/100 g DS kernels, such as 1-10,000 HUT/100 g DS kernels, preferably 300-8,000 HUT/100 g DS kernels, especially 3,000-6,000 HUT/100 g DS kernels, or 4,000-20,000 HUT/100 g DS kernels acidic protease, preferably 5,000-10,000 HUT/100 g, especially from 6,000-16,500 HUT/100 g DS kernels.

Cellulolytic Compositions

In an embodiment, the cellulolytic composition comprises a beta-xylosidase useful according to the invention.

In an embodiment, the cellulolytic composition comprises enzymatic activities aside from or in addition to beta-xylosidase.

In an embodiment the cellulolytic composition is derived from a strain of Trichoderma, such as a strain of Trichoderma reesei; a strain of Humicola, such as a strain of Humicola insolens, and/or a strain of Chrysosporium, such as a strain of Chrysosporium lucknowense.

In a preferred embodiment the cellulolytic composition is derived from a strain of Trichoderma reesei.

The cellulolytic composition may comprise one or more of the following polypeptides, including enzymes: GH61 polypeptide having cellulolytic enhancing activity, beta-glucosidase, beta-xylosidase, CBHI and CBHII, endoglucanase, xylanase, or a mixture of two, three, or four thereof.

In an embodiment the cellulolytic composition comprises a GH61 polypeptide having cellulolytic enhancing activity and a beta-glucosidase.

In an embodiment the cellulolytic composition comprises a GH61 polypeptide having cellulolytic enhancing activity and a beta-xylosidase.

In an embodiment, the cellulolytic composition comprises a GH61 polypeptide having cellulolytic enhancing activity and an endoglucanase.

In an embodiment, the cellulolytic composition comprises a GH61 polypeptide having cellulolytic enhancing activity and a xylanase.

In an embodiment, the cellulolytic composition comprises a GH61 polypeptide having cellulolytic enhancing activity, an endoglucanase, and a xylanase.

In an embodiment the cellulolytic composition comprises a GH61 polypeptide having cellulolytic enhancing activity, a beta-glucosidase, and a beta-xylosidase. In an embodiment the cellulolytic composition comprises a GH61 polypeptide having cellulolytic enhancing activity, a beta-glucosidase, and an endoglucanase. In an embodiment the cellulolytic composition comprises a GH61 polypeptide having cellulolytic enhancing activity, a beta-glucosidase, and a xylanase.

In an embodiment the cellulolytic composition comprises a GH61 polypeptide having cellulolytic enhancing activity, a beta-xylosidase, and an endoglucanase. In an embodiment the cellulolytic composition comprises a GH61 polypeptide having cellulolytic enhancing activity, a beta-xylosidase, and a xylanase.

In an embodiment the cellulolytic composition comprises a GH61 polypeptide having cellulolytic enhancing activity, a beta-glucosidase, a beta-xylosidase, and an endoglucanase. In an embodiment the cellulolytic composition comprises a GH61 polypeptide having cellulolytic enhancing activity, a beta-glucosidase, a beta-xylosidase, and a xylanase. In an embodiment the cellulolytic composition comprises a GH61 polypeptide having cellulolytic enhancing activity, a beta-glucosidase, an endoglucanase, and a xylanase.

In an embodiment the cellulolytic composition comprises a GH61 polypeptide having cellulolytic enhancing activity, a beta-xylosidase, an endoglucanase, and a xylanase.

In an embodiment the cellulolytic composition comprises a GH61 polypeptide having cellulolytic enhancing activity, a beta-glucosidase, a beta-xylosidase, an endoglucanase, and a xylanase.

In an embodiment the endoglucanase is an endoglucanase I.

In an embodiment the endoglucanase is an endoglucanase II.

In an embodiment, the cellulolytic composition comprises a GH61 polypeptide having cellulolytic enhancing activity, an endoglucanase I, and a xylanase.

In an embodiment, the cellulolytic composition comprises a GH61 polypeptide having cellulolytic enhancing activity, an endoglucanase II, and a xylanase.

In another embodiment the cellulolytic composition comprises a GH61 polypeptide having cellulolytic enhancing activity, a beta-glucosidase, and a CBHI.

In another embodiment the cellulolytic composition comprises a GH61 polypeptide having cellulolytic enhancing activity, a beta-glucosidase, a CBHI and a CBHII.

The cellulolytic composition may further comprise one or more enzymes selected from the group consisting of an esterase, an expansin, a laccase, a ligninolytic enzyme, a pectinase, a peroxidase, a protease, a swollenin, and a phytase.

GH61 Polypeptide having Cellulolytic Enhancing Activity

The cellulolytic composition may in one embodiment comprise one or more GH61 polypeptide having cellulolytic enhancing activity.

In one embodiment GH61 polypeptide having cellulolytic enhancing activity, is derived from the genus Thermoascus, such as a strain of Thermoascus aurantiacus, such as the one described in WO 2005/074656 as SEQ ID NO: 2; or SEQ ID NO: 1 herein, or a GH61 polypeptide having cellulolytic enhancing activity having at least 80%, such as at least 85%, such such as at least 90%, preferably 95%, such as at least 96%, such as 97%, such as at least 98%, such as at least 99% identity to SEQ ID NO: 2 in WO 2005/074656 or SEQ ID NO: 1 herein. In one aspect, the protease differs by up to 10 amino acids, e.g., 1, 2, 3, 4, 5, 6, 7, 8, 9, or 10, from the mature polypeptide of SEQ ID NO: 1. In another embodiment, the present invention relates to variants of the mature polypeptide of SEQ ID NO: 1 comprising a substitution, deletion, and/or insertion at one or more (e.g., several) positions. In an embodiment, the number of amino acid substitutions, deletions and/or insertions introduced into the mature polypeptide of SEQ ID NO: 1 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

In one embodiment, the GH61 polypeptide having cellulolytic enhancing activity, is derived from a strain derived from Penicillium, such as a strain of Penicillium emersonii, such as the one disclosed in WO 2011/041397 or SEQ ID NO: 2 herein, or a GH61 polypeptide having cellulolytic enhancing activity having at least 80%, such as at least 85%, such such as at least 90%, preferably 95%, such as at least 96%, such as 97%, such as at least 98%, such as at least 99% identity to SEQ ID NO: 2 in WO 2011/041397 or SEQ ID NO: 2 herein. In one aspect, the protease differs by up to 10 amino acids, e.g., 1, 2, 3, 4, 5, 6, 7, 8, 9, or 10, from the mature polypeptide of SEQ ID NO: 2. In another embodiment, the present invention relates to variants of the mature polypeptide of SEQ ID NO: 2 comprising a substitution, deletion, and/or insertion at one or more (e.g., several) positions. In an embodiment, the number of amino acid substitutions, deletions and/or insertions introduced into the mature polypeptide of SEQ ID NO: 2 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

In one embodiment the GH61 polypeptide having cellulolytic enhancing activity is derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigatus, such as the one described in WO 2010/138754 as SEQ ID NO: 2, or a GH61 polypeptide having cellulolytic enhancing activity having at least 80%, such as at least 85%, such such as at least 90%, preferably 95%, such as at least 96%, such as 97%, such as at least 98%, such as at least 99% identity thereto.

Endoglucanase

In one embodiment, the cellulolytic composition comprises an endoglucanase, such as an endoglucanase I or endoglucanase II.

Examples of bacterial endoglucanases that can be used in the processes of the present invention, include, but are not limited to, an Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of fungal endoglucanases that can be used in the present invention, include, but are not limited to, a Trichoderma reesei endoglucanase I (Penttila et al., 1986, Gene 45: 253-263, Trichoderma reesei Cel7B endoglucanase I (GENBANK™ accession no. M15665), Trichoderma reesei endoglucanase II (Saloheimo, et al., 1988, Gene 63:11-22), Trichoderma reesei Cel5A endoglucanase II (GENBANK™ accession no. M19373), Trichoderma reesei endoglucanase III (Okada et al., 1988, Appl. Environ. Microbiol. 64: 555-563, GENBANK™ accession no. AB003694), Trichoderma reesei endoglucanase V (Saloheimo et al., 1994, Molecular Microbiology 13: 219-228, GENBANK™ accession no. Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Erwinia carotovara endoglucanase (Saarilahti et al., 1990, Gene 90: 9-14), Fusarium oxysporum endoglucanase (GENBANK™ accession no. L29381), Humicola grisea var. thermoidea endoglucanase (GENBANK™ accession no. AB003107), Melanocarpus albomyces endoglucanase (GENBANK™ accession no. MAL515703), Neurospora crassa endoglucanase (GENBANK™ accession no. XM 324477), Humicola insolens endoglucanase V, Myceliophthora thermophila CBS 117.65 endoglucanase, basidiomycete CBS 495.95 endoglucanase, basidiomycete CBS 494.95 endoglucanase, Thielavia terrestris NRRL 8126 CEL6B endoglucanase, Thielavia terrestris NRRL 8126 CEL6C endoglucanase, Thielavia terrestris NRRL 8126 CEL7C endoglucanase, Thielavia terrestris NRRL 8126 CEL7E endoglucanase, Thielavia terrestris NRRL 8126 CEL7F endoglucanase, Cladorrhinum foecundissimum ATCC 62373 CEL7A endoglucanase, and Trichoderma reesei strain No. VTT-D-80133 endoglucanase (GENBANK™ accession no. M15665).

In one embodiment, the endoglucanase is an endoglucanase II, such as one derived from Trichoderma, such as a strain of Trichoderma reesei, such as the one described in WO 2011/057140 as SEQ ID NO: 22; or SEQ ID NO: 3 herein, or an endoglucanase having at least 80%, such as at least 85%, such such as at least 90%, preferably 95%, such as at least 96%, such as 97%, such as at least 98%, such as at least 99% identity to SEQ ID NO: 22 in WO 2011/057140 or SEQ ID NO: 3 herein. In one aspect, the protease differs by up to 10 amino acids, e.g., 1, 2, 3, 4, 5, 6, 7, 8, 9, or 10, from the mature polypeptide of SEQ ID NO: 3. In another embodiment, the present invention relates to variants of the mature polypeptide of SEQ ID NO: 3 comprising a substitution, deletion, and/or insertion at one or more (e.g., several) positions. In an embodiment, the number of amino acid substitutions, deletions and/or insertions introduced into the mature polypeptide of SEQ ID NO: 3 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

Xylanase

In one embodiment, the cellulolytic composition comprises a xylanase. In a preferred aspect, the xylanase is a Family 10 xylanase.

Examples of xylanases useful in the processes of the present invention include, but are not limited to, xylanases from Aspergillus aculeatus (GeneSeqP:AAR63790; WO 94/21785), Aspergillus fumigatus (WO 2006/078256), Penicillium pinophilum (WO 2011/041405), Penicilliurn sp. (WO 2010/126772), Thielavia terrestris NRRL 8126 (WO 2009/079210), and Trichophaea saccata GH10 (WO 2011/057083).

In one embodiment the GH10 xylanase is derived from the genus Aspergillus, such as a strain of Aspergillus aculeatus, such as the one described in WO 94/021785 as SEQ ID NO: 5 (referred to as Xyl II) or SEQ ID NO: 4 herein, or a GH10 xylanase having at least 80%, such as at least 85%, such such as at least 90%, preferably 95%, such as at least 96%, such as 97%, such as at least 98%, such as at least 99% identity to SEQ ID NO: 5 in WO 94/021785 or SEQ ID NO: 4 herein. In one aspect, the protease differs by up to 10 amino acids, e.g., 1, 2, 3, 4, 5, 6, 7, 8, 9, or 10, from the mature polypeptide of SEQ ID NO: 4. In another embodiment, the present invention relates to variants of the mature polypeptide of SEQ ID NO: 4 comprising a substitution, deletion, and/or insertion at one or more (e.g., several) positions. In an embodiment, the number of amino acid substitutions, deletions and/or insertions introduced into the mature polypeptide of SEQ ID NO: 4 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

In one embodiment the GH10 xylanase is derived from the genus Aspergillus, such as a strain of Aspergillus fumigatus, such as the one described as SEQ ID NO: 6 in WO 2006/078256 as Xyl III; or SEQ ID NO: 5 herein, or a GH10 xylanase having at least 80%, such as at least 85%, such such as at least 90%, preferably 95%, such as at least 96%, such as 97%, such as at least 98%, such as at least 99% identity to SEQ ID NO: 6 (Xyl III) in WO 2006/078256 or SEQ ID NO: 5 herein. In one aspect, the protease differs by up to 10 amino acids, e.g., 1, 2, 3, 4, 5, 6, 7, 8, 9, or 10, from the mature polypeptide of SEQ ID NO: 5. In another embodiment, the present invention relates to variants of the mature polypeptide of SEQ ID NO: 5 comprising a substitution, deletion, and/or insertion at one or more (e.g., several) positions. In an embodiment, the number of amino acid substitutions, deletions and/or insertions introduced into the mature polypeptide of SEQ ID NO: 5 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function.

Beta-Glucosidase

The cellulolytic composition may in one embodiment comprise one or more beta-glucosidase. The beta-glucosidase may in one embodiment be one derived from a strain of the genus Aspergillus, such as Aspergillus oryzae, such as the one disclosed in WO 2002/095014 or the fusion protein having beta-glucosidase activity disclosed, e.g., as SEQ ID NO: 74 or 76 in WO 2008/057637, or Aspergillus fumigatus, such as one disclosed as SEQ ID NO: 2 in WO 2005/047499 or an Aspergillus fumigatus beta-glucosidase variant, such as one disclosed in PCT application PCT/US11/054185 or WO 2012/044915 (or U.S. provisional application No. 61/388,997), such as one with the following substitutions: F100D, S283G, N456E, F512Y.

In one embodiment the beta-glucosidase is derived from the genus Aspergillus, such as a strain of Aspergillus fumigatus, such as the one described as SEQ ID NO: 2 in WO 2005/047499, or a beta-glucosidase having at least 80%, such as at least 85%, such such as at least 90%, preferably 95%, such as at least 96%, such as 97%, such as at least 98%, such as at least 99% identity thereto.

In one embodiment the beta-glucosidase is derived from the genus Aspergillus, such as a strain of Aspergillus fumigatus, such as the one described as SEQ ID NO: 2 in WO 2005/047499 or in WO 2012/044915, or a beta-glucosidase having at least 80%, such as at least 85%, such such as at least 90%, preferably 95%, such as at least 96%, such as 97%, such as at least 98%, such as at least 99% identity thereto.

Cellobiohydrolase I

The cellulolytic composition may in one embodiment may comprise one or more CBH I (cellobiohydrolase I). In one embodiment the cellulolytic composition comprises a cellobiohydrolase I (CBHI), such as one derived from a strain of the genus Aspergillus, such as a strain of Aspergillus fumigatus, such as the Cel7A CBHI disclosed as SEQ ID NO: 2 in WO 2011/057140, or a strain of the genus Trichoderma, such as a strain of Trichoderma reesei.

In one embodiment the cellobiohydrolyase I is derived from the genus Aspergillus, such as a strain of Aspergillus fumigatus, such as the one described as SEQ ID NO: 6 in WO 2011/057140, or a CBHI having at least 80%, such as at least 85%, such such as at least 90%, preferably 95%, such as at least 96%, such as 97%, such as at least 98%, such as at least 99% identity thereto.

Cellobiohydrolase II

The cellulolytic composition may in one embodiment comprise one or more CBH II (cellobiohydrolase II). In one embodiment the cellobiohydrolase II (CBHII), such as one derived from a strain of the genus Aspergillus, such as a strain of Aspergillus fumigatus, or a strain of the genus Trichoderma, such as Trichoderma reesei, or a strain of the genus Thielavia, such as a strain of Thielavia terrestris, such as cellobiohydrolase II CEL6A from Thielavia terrestris.

In one embodiment the cellobiohydrolyase II is derived from the genus Aspergillus, such as a strain of Aspergillus fumigatus, such as the one described as SEQ ID NO: 18 in WO 2011/057140, or a CBHII having at least 80%, such as at least 85%, such such as at least 90%, preferably 95%, such as at least 96%, such as 97%, such as at least 98%, such as at least 99% identity thereto.

Exemplary Cellulolytic Compositions

As mentioned above the cellulolytic composition may comprise a number of different polypeptides, such as enzymes.

In an embodiment, the cellulolytic composition comprises a Trichoderma reesei cellulolytic enzyme composition containing Aspergillus oryzae beta-glucosidase fusion protein (e.g., SEQ ID NO: 74 or 76 in WO 2008/057637) and Thermoascus aurantiacus GH61A polypeptide (e.g., SEQ ID NO: 2 WO 2005/074656).

In an embodiment, the cellulolytic composition comprises a blend of an Aspergillus aculeatus GH10 xylanase (e.g., as SEQ ID NO: 5 (XYL II) in WO 94/021785) and a Trichoderma reesei cellulolytic enzyme composition containing Aspergillus fumigatus beta-glucosidase (e.g., SEQ ID NO: 2 in WO 2005/047499) and Thermoascus aurantiacus GH61A polypeptide (e.g., SEQ ID NO: 2 in WO 2005/074656).

In an embodiment, the cellulolytic composition comprises a blend of an Aspergillus fumigatus GH10 xylanase (e.g., SEQ ID NO: 6 (Xyl III) in WO 2006/078256) and Aspergillus fumigatus beta-xylosidase (e.g., SEQ ID NO: 206 in WO 2011/057140) with a Trichoderma reesei cellulolytic enzyme composition containing Aspergillus fumigatus cellobiohydrolase I (e.g., SEQ ID NO: 6 in WO 2011/057140), Aspergillus fumigatus cellobiohydrolase II (e.g., SEQ ID NO: 18 in WO 2011/057140), Aspergillus fumigatus beta-glucosidase variant (e.g., one having F100D, S283G, N456E, F512Y substitutions disclosed in WO 2012/044915), and Penicillium sp. (emersonii) GH61 polypeptide (e.g., SEQ ID NO: 2 WO 2011/041397).

In an embodiment the cellulolytic composition comprises a Trichoderma reesei cellulolytic enzyme composition, further comprising Thermoascus aurantiacus GH61A polypeptide having cellulolytic enhancing activity (e.g., SEQ ID NO: 2 in WO 2005/074656) and Aspergillus oryzae beta-glucosidase fusion protein (e.g., SEQ ID NO: 74 or 76 in WO 2008/057637).

In another embodiment the cellulolytic composition comprises a Trichoderma reesei cellulolytic enzyme composition, further comprising Thermoascus aurantiacus GH61A polypeptide having cellulolytic enhancing activity (e.g., SEQ ID NO: 2 in WO 2005/074656) and Aspergillus fumigatus beta-glucosidase (e.g., SEQ ID NO: 2 of WO 2005/047499).

In another embodiment the cellulolytic composition comprises a Trichoderma reesei cellulolytic enzyme composition, further comprising Penicillium emersonii GH61A polypeptide having cellulolytic enhancing activity disclosed as SEQ ID NO: 2 in WO 2011/041397, Aspergillus fumigatus beta-glucosidase (e.g., SEQ ID NO: 2 of WO 2005/047499) or a variant thereof with the following substitutions: F100D, S283G, N456E, F512Y.

The enzyme composition of the present invention may be in any form suitable for use, such as, for example, a crude fermentation broth with or without cells removed, a cell lysate with or without cellular debris, a semi-purified or purified enzyme composition, or a host cell, e.g., Trichoderma host cell, as a source of the enzymes.

The enzyme composition may be a dry powder or granulate, a non-dusting granulate, a liquid, a stabilized liquid, or a stabilized protected enzyme. Liquid enzyme compositions may, for instance, be stabilized by adding stabilizers such as a sugar, a sugar alcohol or another polyol, and/or lactic acid or another organic acid according to established processes.

According to the invention an effective amount of one or more of the following activities may also be present or added during treatment of the kernels: pentosanase, pectinase, arabinanase, arabinofurasidase, xyloglucanase, phytase activity.

It is believed that after the division of the kernels into finer particles the enzyme(s) can act more directly and thus more efficiently on cell wall and protein matrix of the kernels. Thereby the starch is washed out more easily in the subsequent steps.

Enzymatic Amount

Enzymes may be added in an effective amount, which can be adjusted according to the practitioner and particular process needs. In general, enzyme may be present in an amount of 0.0001-1 mg enzyme protein per g dry solids (DS) kernels, such as 0.001-0.1 mg enzyme protein per g DS kernels. In particular embodiments, the enzyme may be present in an amount of, e.g., 1 μg, 2.5 μg, 5 μg, 10 μg, 20 μg, 25 μg, 50 μg, 75 μg, 100 μg, 125 μg, 150 μg, 175 μg, 200 μg, 225 μg, 250 μg, 275 μg, 300 μg, 325 μg, 350 μg, 375 μg, 400 μg, 450 μg, 500 μg, 550 μg, 600 μg, 650 μg, 700 μg, 750 μg, 800 μg, 850 μg, 900 μg, 950 μg, 1000 μg enzyme protein per g DS kernels.

### Preferred Embodiments

The following embodiments of the invention are exemplary.

1. A process for treating crop kernels, comprising the steps of:
a) soaking kernels in water to produce soaked kernels;
b) grinding the soaked kernels; and
c) treating the soaked kernels in the presence of an effective amount of a beta-xylosidase;

wherein step c) is performed before, during or after step b).

2. The process of any of the preceding embodiments, wherein the beta-xylosidase is present in an amount of 0.0001-1 mg enzyme protein per g dry solids (DS) kernels, such as 0.001-0.1 mg enzyme protein per g DS kernels.

3. The process of any of the preceding embodiments, wherein the beta-xylosidase is present in an amount of, e.g., 1 μg, 2.5 μg, 5 μg, 10 μg, 20 μg, 25 μg, 50 μg, 75 μg, 100 μg, 125 μg, 150 μg, 175 μg, 200 μg, 225 μg, 250 μg, 275 μg, 300 μg, 325 μg, 350 μg, 375 μg, 400 μg, 450 μg, 500 μg, 550 μg, 600 μg, 650 μg, 700 μg, 750 μg, 800 μg, 850 μg, 900 μg, 950 μg, 1000 μg enzyme protein per g DS kernels.

4. The process of embodiment 1, further comprising treating the soaked kernels in the presence of a protease.

5. The process of any of the preceding embodiments, further comprising treating the soaked kernels in the presence of a cellulolytic composition.

6. The process of any of the preceding embodiments, further comprising treating the soaked kernels in the presence of an enzyme selected from the group consisting of an endoglucanase, a xylanase, a cellobiohydrolase I, a cellobiohydrolase II, a GH61 polypeptide, or a combination thereof.

7. The process of any of the preceding embodiments, further comprising treating the soaked kernels in the presence of an endoglucanase.

8. The process of any of the preceding embodiments, further comprising treating the soaked kernels in the presence of a xylanase.

9. The process of any of the preceding embodiments, further comprising treating the soaked kernels in the presence of a cellulolytic composition.

10. The process of any of the preceding embodiments, wherein the cellulolytic composition comprises a Trichoderma reesei cellulolytic enzyme composition containing Aspergillus oryzae beta-glucosidase fusion protein (e.g., SEQ ID NO: 74 or 76 in WO 2008/057637) and Thermoascus aurantiacus GH61A polypeptide (e.g., SEQ ID NO: 2 in WO 2005/074656).

11. The process of any of the preceding embodiments, wherein the cellulolytic composition comprises a blend of an Aspergillus aculeatus GH10 xylanase (e.g., SEQ ID NO: 5 (Xyl II) in WO 94/021785) and a Trichoderma reesei cellulolytic enzyme composition containing Aspergillus fumigatus beta-glucosidase (e.g., SEQ ID NO: 2 in WO 2005/047499) and Thermoascus aurantiacus GH61A polypeptide (e.g., SEQ ID NO: 2 in WO 2005/074656).

12. The process of any of the preceding embodiments, wherein the cellulolytic composition comprises a blend of an Aspergillus fumigatus GH10 xylanase (e.g., SEQ ID NO: 6 (Xyl III) in WO 2006/078256) and Aspergillus fumigatus beta-xylosidase (e.g., SEQ ID NO: 16 in WO 2013/028928—see Examples 16 and 17 or SEQ ID NO: 206 in WO 2011/057140) with a Trichoderma reesei cellulolytic enzyme composition containing Aspergillus fumigatus cellobiohydrolase I (e.g., SEQ ID NO: 6 in WO 2011/057140), Aspergillus fumigatus cellobiohydrolase II (e.g., SEQ ID NO: 18 in WO 2011/057140), Aspergillus fumigatus beta-glucosidase variant (e.g., one having F100D, S283G, N456E, F512Y substitutions described in WO 2012/044915), and Penicillium sp. (emersonii) GH61 polypeptide (e.g., SEQ ID NO: 2 in WO 2011/041397).

13. The process of any of the preceding embodiments, wherein the cellulolytic composition comprises a Trichoderma reesei cellulolytic enzyme composition, further comprising Thermoascus aurantiacus GH61A polypeptide having cellulolytic enhancing activity (e.g., SEQ ID NO: 2 in WO 2005/074656) and Aspergillus oryzae beta-glucosidase fusion protein (e.g., SEQ ID NO: 74 or 76 in WO 2008/057637).

14. The process of any of the preceding embodiments, wherein the cellulolytic composition comprises a Trichoderma reesei cellulolytic enzyme composition, further comprising Thermoascus aurantiacus GH61A polypeptide having cellulolytic enhancing activity (SEQ ID NO: 2 in WO 2005/074656) and Aspergillus fumigatus beta-glucosidase (SEQ ID NO: 2 of WO 2005/047499).

15. The process of any of the preceding embodiments, wherein the cellulolytic composition comprises a Trichoderma reesei cellulolytic enzyme composition, further comprising Penicillium emersonii GH61A polypeptide having cellulolytic enhancing activity disclosed as SEQ ID NO: 2 in WO 2011/041397, Aspergillus fumigatus beta-glucosidase (SEQ ID NO: 2 of WO 2005/047499) or a variant thereof with the following substitutions: F100D, S283G, N456E, F512Y.

16. The process of any of the preceding embodiments, further comprising treating the kernels with pentosanase, pectinase, arabinanase, arabinofurasidase, xyloglucanase, protease, and/or phytase.

17. The process of any of the preceding embodiments, wherein the kernels are soaked in water for about 2-10 hours, preferably about 3 hours.

18. The process of any of the preceding embodiments, wherein the soaking is carried out at a temperature between about 40° C. and about 60° C., preferably about 50° C.

19. The process of any of the preceding embodiments, wherein the soaking is carried out at acidic pH, preferably about 3-5, such as about 3-4.

20. The process of any of the preceding embodiments, wherein the soaking is performed in the presence of between 0.01-1%, preferably 0.05-0.3%, especially 0.1% SO2 and/or NaHSO3. 1. The process of any of the preceding embodiments, wherein the crop kernels are from corn (maize), rice, barley, sorghum bean, or fruit hulls, or wheat.

22. Use of a beta-xylosidase to enhance the wet milling benefit of one or more enzymes.

23. The use of any of the preceding embodiments, wherein the beta-xylosidase is present in an amount of 0.0001-1 mg enzyme protein per g dry solids (DS) kernels, such as 0.001-0.1 mg enzyme protein per g DS kernels.

24. The use of any of the preceding embodiments, wherein the beta-xylosidase is present in an amount of, e.g., 1 μg, 2.5 μg, 5 μg, 10 μg, 20 μg, 25 μg, 50 μg, 75 μg, 100 μg, 125 μg, 150 μg, 175 μg, 200 μg, 225 μg, 250 μg, 275 μg, 300 μg, 325 μg, 350 μg, 375 μg, 400 μg, 450 μg, 500 μg, 550 μg, 600 μg, 650 μg, 700 μg, 750 μg, 800 μg, 850 μg, 900 μg, 950 μg, 1000 μg enzyme protein per g DS kernels.

25. The use of any of the preceding embodiments, further comprising treating the soaked kernels in the presence of a cellulolytic composition.

26. The use of any of the preceding embodiments, wherein the cellulolytic composition comprises a Trichoderma reesei cellulolytic enzyme composition containing Aspergillus oryzae beta-glucosidase fusion protein (e.g., SEQ ID NO: 74 or 76 in WO 2008/057637) and Thermoascus aurantiacus GH61A polypeptide (e.g., SEQ ID NO: 2 in WO 2005/074656).

27. The use of any of the preceding embodiments, wherein the cellulolytic composition comprises a blend of an Aspergillus aculeatus GH10 xylanase (e.g., SEQ ID NO: 5 (Xyl II) in WO 94/021785) and a Trichoderma reesei cellulolytic enzyme composition containing Aspergillus fumigatus beta-glucosidase (e.g., SEQ ID NO: 2 in WO 2005/047499) and Thermoascus aurantiacus GH61A polypeptide (e.g., SEQ ID NO: 2 in WO 2005/074656).

28. The use of any of the preceding embodiments, wherein the cellulolytic composition comprises a blend of an Aspergillus fumigatus GH10 xylanase (e.g., SEQ ID NO: 6 (Xyl III) in WO 2006/078256) and Aspergillus fumigatus beta-xylosidase (e.g., SEQ ID NO: 16 in WO 2013/028928—see Examples 16 and 17 or SEQ ID NO: 206 in WO 2011/057140) with a Trichoderma reesei cellulolytic enzyme composition containing Aspergillus fumigatus cellobiohydrolase I (e.g., SEQ ID NO: 6 in WO 2011/057140), Aspergillus fumigatus cellobiohydrolase II (e.g., SEQ ID NO: 18 in WO 2011/057140), Aspergillus fumigatus beta-glucosidase variant (e.g., one having F100D, S283G, N456E, F512Y substitutions described in WO 2012/044915), and Penicillium sp. (emersonii) GH61 polypeptide (e.g., SEQ ID NO: 2 in WO 2011/041397).

29. The use of any of the preceding embodiments, wherein the cellulolytic composition comprises a Trichoderma reesei cellulolytic enzyme composition, further comprising Thermoascus aurantiacus GH61A polypeptide having cellulolytic enhancing activity (e.g., SEQ ID NO: 2 in WO 2005/074656) and Aspergillus oryzae beta-glucosidase fusion protein (e.g., SEQ ID NO: 74 or 76 in WO 2008/057637).

30. The use of any of the preceding embodiments, wherein the cellulolytic composition comprises a Trichoderma reesei cellulolytic enzyme composition, further comprising Thermoascus aurantiacus GH61A polypeptide having cellulolytic enhancing activity (SEQ ID NO: 2 in WO 2005/074656) and Aspergillus fumigatus beta-glucosidase (SEQ ID NO: in WO 2005/047499).

31. The use of any of the preceding embodiments, wherein the cellulolytic composition comprises a Trichoderma reesei cellulolytic enzyme composition, further comprising Penicillium emersonii GH61A polypeptide having cellulolytic enhancing activity disclosed, e.g., as SEQ ID NO: 2 in WO 2011/041397, Aspergillus fumigatus beta-glucosidase (SEQ ID NO: 2 in WO 2005/047499) or a variant thereof with the following substitutions: F100D, S283G, N456E, F512Y (see WO2012/044915).

32. The use of any of the preceding embodiments, further comprising treating the kernels with pentosanase, pectinase, arabinanase, arabinofurasidase, xyloglucanase, and/or phytase.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties.

### EXAMPLES

Materials and Methods

Enzymes:

Protease I: Acidic protease from Aspergillus aculeatus, CBS 101.43 disclosed in WO 95/02044.

Protease A: Aspergillus oryzae aspergillopepsin A, disclosed in Gene, vol. 125, issue 2, pages 195-198 (30 Mar. 1993).

Protease B: A metalloprotease from Thermoascus aurantiacus (AP025) having the acid sequence shown as SEQ ID NO: 2 in WO2003/048353A1 and available from Novozymes A/S, Denmark.

Protease C: Rhizomucor miehei derived aspartic endopeptidase produced in Aspergillus oryzae (Novoren™)

Cellulase A: A blend of an Aspergillus aculeatus GH10 xylanase (SEQ ID NO: 5 in WO 1994/021785 or SEQ ID NO: 4 herein) and a Trichoderma reesei cellulolytic enzyme composition containing Aspergillus fumigatus beta-glucosidase (SEQ ID NO: 2 in WO 2005/047499) and Thermoascus aurantiacus GH61A polypeptide (SEQ ID NO: 2 in WO 2005/074656).

Cellulase B: A Trichoderma reesei cellulolytic enzyme composition containing Aspergillus oyrzae beta-glucosidase fusion protein (WO 2008/057637) and Thermoascus aurantiacus GH61A polypeptide (SEQ ID NO: 2 in WO 2005/074656).

Cellulase C: A blend of an Aspergillus fumigatus GH10 xylanase (SEQ ID NO: 6 (Xyl III) in WO 2006/078256) and Aspergillus fumigatus beta-xylosidase (SEQ ID NO: 16 in WO 2013/028928—see Examples 16 and 17) with a Trichoderma reesei cellulolytic enzyme composition containing Aspergillus fumigatus cellobiohydrolyase I (SEQ ID NO: 6 in WO 2011/057140), Aspergillus fumigatus cellobiohydrolase II (SEQ ID NO: 18 in WO 2011/057140), Aspergillus fumigatus beta-glucosidase variant (with F100D, S283G, N456E, F512Y substitutions disclosed in WO 2012/044915), and Penicillium sp. (emersonii) GH61 polypeptide (SEQ ID NO: 2 in WO 2011/041397).

Cellulase D: Aspergillus aculeatus GH10 xylanase (SEQ ID NO: 5 (Xyl II) in WO 1994/021785).

Cellulase E: A Trichoderma reesei cellulolytic enzyme composition containing Aspergillus aculeatus GH10 xylanase (SEQ ID NO: 5 (Xyl II) WO 1994/021785).

Cellulase F: A Trichoderma reesei cellulolytic enzyme composition containing Aspergillus fumigatus GH10 xylanase (SEQ ID NO: 6 (Xyl III) in WO 2006/078256) and Aspergillus fumigatus beta-xylosidase (SEQ ID NO: 16 in WO 2013/028928).

Cellulase G: A cellulolytic enzyme composition containing Aspergillus aculeatus Family 10 xylanase (SEQ ID NO: 5 (Xyl II in WO1994/021785) and cellulolytic composition derived from Trichoderma reesei RutC30.

Cellulase H: Aspergillus aculeatus Family 10 xylanase (SEQ ID NO: 5 (Xyl II in WO1994/021785).

Cellulase I: Trichoderma reesei endoglucanase EGI.

Methods

Determination of Protease HUT Activity:

1 HUT is the amount of enzyme which, at 40° C. and pH 4.7 over 30 minutes forms a hydrolysate from digesting denatured hemoglobin equivalent in absorbancy at 275 nm to a solution of 1.10 μg/ml tyrosine in 0.006 N HCl which absorbancy is 0.0084. The denatured hemoglobin substrate is digested by the enzyme in a 0.5 M acetate buffer at the given conditions. Undigested hemoglobin is precipitated with trichloroacetic acid and the absorbance at 275 nm is measured of the hydrolysate in the supernatant.

### Example 1

### Wet Milling in the Presence of Cellulase F Compared to Cellulase H

Two experiments were performed in which three treatments of corn were put through a simulated corn wet milling process according to the procedure below. In each experiment, two treatments involved application of enzyme (Steeps B, and C) whereas one treatment was enzyme-free (Steep A). In Experiment 1, the design of experiment was as shown in Table 1:

TABLE 1
Experimental Design For Experiment 1

| Steep A | Steep B | Steep C |
|---|---|---|
| Enzyme-Free | Cellulase H - | Cellulase H - |
| Control | 50 μg/g Dose | 250 μg/g Dose |

Experiment 2 was designed as shown in Table 2:

TABLE 2
Experimental Design Experiment 2

| Steep A | Steep B | Steep C |
|---|---|---|
| Enzyme-Free | Cellulase F - | Cellulase F - |
| Control | 50 μg/g Dose | 250 μg/g Dose |

In both experiments, the amounts of enzyme protein applied for the high doses were identical to each other. The same was true for all of the low doses. High doses utilized a dose level of 250 μg per gram of corn dry substance. The low dose level was 50 μg per gram of corn dry substance. For the enzyme treated steeps (Steeps B and C), a steep solution containing 0.06% (w/v) SO₂ and 0.5% (w/v) lactic acid was assembled. 100 grams of dry regular (yellow dent) corn was cleaned to remove the broken kernels and put into 200 mL of the steep water described above for each flask. All flasks were then put into an orbital air heated shaker machine which was set to 52° C. with mild shaking and allowed to mix at this temperature for 16 hours. After 16 hours, all flasks were removed from the air shaker. The enzyme-free control steep (Steep A) was made up in a similar fashion; with the exception being that it was steeped in a 0.15% (w/v) SO₂ solution, and was steeped for 30 hours prior to grinding. The corn mixture was poured over a Buchner funnel to dewater it, and 100 mL of fresh tap water was then added to the original steeping flask and swirled for rinsing purpose. It was then poured over the corn as a wash and captured in the same flask as the original corn draining. The purpose of this washing step was to retain as many of the solubles with the filtrate as possible. The filtrate containing solubles was called “light steep water”. The total light steep water fraction collected was then oven-dried to determine the amount of dry substance present. The drying was done by overnight drying in oven set by 105° C.

The corn was then placed into a Waring Laboratory Blender with the blades reversed (so the leading edge was dull). 200 mL of water was added to the corn in the blender, and the corn was then ground for one minute at low speed setting to facilitate germ release. Once ground, the slurry was transferred back to flasks for enzymatic incubation step. 50 mL fresh water was used to rinse the blender and the wash water was added to the flask as well. The enzyme treatment flasks (Steeps B, and C) were dosed with enzyme and returned to orbital shaker to be incubated at 52° C. for another 4 hours at higher mixing rate.

After incubation, the slurry was transferred to a large beaker for released germ removal. The control steep did not go through this incubation step but was ground and then processed immediately as described below.

For degermination, a slotted spoon was used to gently stir the mixture briefly. After the stirring was stopped, large quantities of germ pieces floated to the surface. These were skimmed off of the liquid surface manually using the slotted spoon. The germ pieces were placed on a US No. 100 (150 μm) screen with a catch pan underneath of it. This process of mixing and skimming was repeated until negligible amounts of germ floated up to the surface for skimming. Inspection of the slurry mash in the slotted spoon also showed no evidence of large germ quantities left in the mixture at this point, so de-germination was stopped. The germ pieces that had been accumulated on the No. 100 screen were then added to a flask where they were combined with 125 mL of fresh water, and swirled to simulate a germ wash tank. The contents of the flask were then poured over the screen again, making sure to tap the flask and fully clear it of germ. The de-germinated slurry in the skimming beaker was then poured back into the blender, and the germ wash water in the catch pan underneath of the screen was used to rinse the germ beaker to the blender. Another 125 mL of fresh water was then used to conduct a second rinse of the beaker and was added to the blender. The washed germ on the screen was oven dried overnight at 105° C. prior to analysis.

The fiber, starch, and gluten slurry that had been de-germinated was then ground in the blender for 3 minutes at high speed. This increased speed was employed to release as much starch and gluten from the fiber as possible. The resulting ground slurry in the blender was screened over a No. 100 vibrating screen (Retsch Model AS200 sieve shaking unit) with a catch pan underneath. The shaking frequency on the Retsch unit was set to roughly 60 HZ. Once filtration had stopped, the starch and gluten filtrate (called “mill starch”) in the catch pan was transferred into a flask until further processing. The fiber on the screen was then slurried in 500 mL of fresh water and then re-poured over the vibrating screen to wash the unbound starch off of the fiber. Again, the starch and gluten filtrate in the catch pan was added to the previous mill starch flask.

The fiber was then washed and screened in this manner three successive times, each time using 240 mL of fresh wash water. This was then followed by a single 125 mL wash while vibrating to achieve maximum starch and gluten liberation from the fiber fraction. After all washings were complete, the fiber was gently pressed on the screen to dewater it before it was transferred to an aluminum weighing pan for oven drying at 105° C. (overnight). All of the filtrate from the washings and pressing was added to the mill starch flask.

The starch and gluten comprising the mill starch were separated using a starch table. The starch table used was a stainless steel u-channel 2.5 cm wide×5 cm deep×305 cm long. The incline of the table was 1″ rise to 66″ run. Slurry was pumped into the raised end of the table at a rate of approximately 48 mL per minute using a peristaltic pump. The gluten runoff was captured in a beaker at the end of the table. It should be noted that the exit end of the table had a stir rod propped up against it to serve as a surface tension breaker, and allow the gluten slurry to flow steadily off of the table where it was collected in a beaker. Once the entire starch gluten slurry had been pumped across the table, 100 mL of fresh water was put in the pump feed flask and pumped onto the table to ensure that all starch had been captured from the feed flask. The flow from the table was allowed to stop completely, and all of the liquid which had flowed off of the end of the table was collected as gluten slurry. The starch left on the table was then washed off the table into a fresh container using 2,500 mL of fresh water. The total volume of gluten solution was measured before gluten filtration. Both the starch and the gluten insolubles were then vacuum filtered. Both fractions were dried in a 105° C. oven for yield measurement. However, they were pre-dried overnight in a 50° C. oven first to remove the bulk of the water from them to minimize gelatinization and incomplete drying. After oven drying, each fraction was weighed to obtain a dry matter weight.

To calculate the solubles generated in the process, gluten filtrate was collected and the total solids content of the filtrate was measured by oven drying a 250 mL portion of the filtrate at 105° C. The total soluble solids content of this fraction was calculated by multiplying the volume of gluten solution by total solids of gluten filtrate.

Tables 3 and 4 below show the product yields (percent of dry solids of each fraction per 100 g dry matter of corn) obtained for control and enzymatic runs in both experiments.

TABLE 3
Fraction yields for the experimental control
and all blends in Experiment 1.

Steep

| | | |
|---|---|---|
| A | B | C |

Treatment

| | Enzyme-Free | Cellulase H - | Cellulase H - |
|---|---|---|---|
| | Control | Low Dose | High Dose |
| Starch | 65.13% | 63.90% | 61.74% |
| Gluten | 8.36% | 9.00% | 10.81% |
| Germ | 7.13% | 6.60% | 6.77% |
| Fiber | 10.86% | 11.69% | 11.36% |
| LSW Solubles | 4.30% | 4.10% | 3.83% |
| Filtrate Solubles | 2.02% | 2.02% | 2.15% |
| Starch + Gluten | 73.48% | 72.90% | 72.56% |

TABLE 4
Fraction yields for the experimental control
and all blends in Experiment 2.

Steep

| | A | B | C |
|---|---|---|---|
| | Enzyme-Free | Cellulase F - | Cellulase F - |
| | Control | Low Dose | High Dose |
| Starch | 64.81% | 63.71% | 65.47% |
| Gluten | 7.84% | 10.01% | 9.05% |
| Germ | 7.55% | 6.93% | 6.65% |
| Fiber | 10.91% | 10.30% | 9.35% |
| LSW Solubles | 4.13% | 3.85% | 3.87% |
| Filtrate Solubles | 2.02% | 2.14% | 2.40% |
| Starch + Gluten | 72.66% | 73.72% | 74.52% |

The starch and gluten yield results obtained for each treatment were divided by that of the respective experimental enzyme-free control. The results of this analysis are shown below in Table 5.

TABLE 5
Starch and Gluten Yields Relative To Experimental
Control For Both Experiments

| | Starch and Gluten Yields |
|---|---|
| Treatment | Relative to Control (%) |
| Cellulase H - Low Dose | 99.2% |
| Cellulase H - High Dose | 98.7% |
| Cellulase F - Low Dose | 101.5% |
| Cellulase F - High Dose | 102.6% |

Table 3 shows that enzymatic treatment using Cellulase H (a xylanase only product) did not improve starch yield or combined starch and gluten yield compared to non-enzymatic treatment.

However yield results from Table 4 and 5 indicate that Cellulase F, which includes a beta xylosidase component in addition to the xylanase component, resulted in higher starch yield and also combined starch and gluten yields than xylanase alone.

### Example 2

### Wet Milling in the Presence of Cellulase F Compared to Cellulase G

Two experiments (designated Experiments 1 and 2) were conducted to compare the performance of Cellulase F, including a beta-xylosidase component, and Cellulase G, including xylanse and cellulase components only, and which does not include a beta-xylosidase component, in which 3 or 5 corn steeps were assembled and ground, respectively, to simulate the industrial corn wet milling process. They were processed individually using the same equipment and methodology. Each experiment included one the conventional steep (experiment 1, steep 1A; experiment 2, steep 2A) and the rest were enzymatic steps (experiment 1 steep 1B, 10 and 1E; experiment 2, steep 2B,2C, 2D and 2E). The various process steps are described below.

The moisture of the corn used in the experiment was determined by loss in weight during oven drying. The corn that was used was weighed and placed in a 105° C. oven for 72 hours. The corn was then re-weighed after oven drying. The loss in weight was used to determine the corn's original solids content.

Steeping: The conventional sample (steep A) was steeped in a 0.15% (w/v) SO2 and a 0.5% (w/v) lactic acid solution for 28 hours prior to milling. The enzymatic sample (steep B to E) was steeped in a 0.06% (w/v) SO2 and 0.5% (w/v) lactic acid solution for 16 hours prior to milling. 100 grams of dry corn was put into 200 mL of the steep water described above. The entire mixture was then put into an orbital air heated shaker machine which was set to 175 RPM at 52° C. and allowed to mix at this temperature for desired time. At the end of the steeping process, the corn mixture was poured over a Buchner funnel for dewatering, and 100 mL of fresh tap water was added to the original steeping flask for rinsing purposes. It was then poured over the corn as a wash and captured in the same flask as the original corn draining. The purpose of this washing step was to retain as many of the solubles with the filtrate as possible. The total light steepwater fraction was placed into a tared flask and oven-dried completely at 105° C. for 24 hours. The flask was weighed post-drying to determine the amount of dry substance present.

First Grind: The corn was then placed into a Waring Laboratory Blender with the blades reversed (so the leading edge was dull). 200 mL of water was added to the corn in the blender, along with the corn rinsewater from above, and the corn was ground for one minute to facilitate germ release. 50 mL of fresh water was used to rinse out the blender and was then poured into the plastic bucket along with the first grind material. Then the slurry was transferred back to each flask and enzymes (1B and 1C, 2B to 2E) were added as shown below in table 6. The flask with corn slurry was transferred to orbital shaker and incubated at 52° C. for 4 hours. After incubation, the slurry was poured out to a 5 L plastic bucket for manual germ removal.

TABLE 6
Experimental design

| | Steeps | | | | | | |
|---|---|---|---|---|---|---|---|
| | 1A, 2A | 1B | 1C | 2B | 2C | 2D | 2E |
| Enzyme Used | Conventional | Cellulase F | Cellulase G | Cellulase F | Cellulase G | Cellulase F | Cellulase G |
| | No-Enzyme | | | | | | |
| μg EP/g dry | 0 | 125 | 125 | 50 | 50 | 25 | 25 |
| corn | | | | | | | |

The control was ground and treated as described below immediately after 28 hours steeping without 2^{nd} time incubation.

De-germination: A slotted spoon was used to gently stir the mixture briefly. After the stirring was stopped, large quantities of germ pieces floated to the surface. These were skimmed off of the liquid surface using the slotted spoon.

The germ pieces were placed on a US No. 100 screen with a catch pan underneath of it. This process of mixing and skimming was repeated until negligible germ floated up to the surface for skimming. Inspection of the settled slurry mash in the slotted spoon also showed no evidence of large germ quantities left in the mixture at this point, so de-germination was stopped.

The germ pieces that had been accumulated on the No. 100 screen were transferred to a small beaker and swirled around with 125 mL of fresh tapwater to wash as much of the starch off of the germ as possible.

The germ and water in the beaker were poured back over the 100 mesh screen for dewatering. The degerminated slurry in the bucket was then poured back into the blender for a second grind. The water that passed through the 100 mesh screen from the 1st germ wash was then used to rinse the plastic bucket into the blender. A second 125 mL aliquot of tapwater was then poured over the germ pieces on the screen to facilitate further washing. This water was collected again in the catch pan and used as a second rinse of the plastic bucket into the blender. The germ on the screen was then pressed with a spatula and transferred to a tared weight pan and oven dried for 24 hours at 105° C. before analysis.

Second grind: The fiber, starch, and gluten slurry that had been de-germinated was then ground in the blender for 3 minutes with the high speed. This increased speed was employed to release as much starch and gluten from the fiber as possible.

Fiber washing: With the second grind complete, the slurry in the blender was screened over a No. 100 vibrating screen (Retsch Model A200 sieve shaking unit). The shaking frequency on the Retsch unit was set to roughly 60HZ. Once filtration had stopped, the starch and gluten filtrate portion was transferred into a flask for storage until tabling. 500 mL of fresh water was then used to rinse the blender after the second grind into a plastic bucket. The fiber on the top of the fiber screen was then added to the plastic bucket, swirled around in the 500 mL of fresh water and then re-screened. The filtrate from this washing was then transferred to the storage flask along with the first batch of filtrate.

The fiber was then washed and screened in this manner three successive times, each time using 240 mL of fresh wash water. This was then followed by a single 125 mL wash while vibrating to achieve maximum starch and gluten liberation from the fiber fraction. After all washings were complete, the fiber was gently pressed on the screen to dewater it before it was transferred to a tared aluminum weighing pan for oven drying at 105° C. for 24 hours prior to weighing.

All of the filtrate from the washings and pressing was added to the storage flask, yielding a total starch and gluten slurry volume of approximately 1,800 mL.

The starch and gluten slurry was then vacuum filtered through a Buchner Funnel through a Whatman filter paper before being oven dried. The total filtrate volume from the vacuum flask was measured. 250 ml filtrate was transferred to a plastic bottle for oven drying at 105° C. for 48 hours. The total soluble solid content of this fraction was calculated by multiplying the volume of gluten solution by total solids of gluten filtrate. The filter cake was transferred to a stainless steel dish to dry overnight fist at 50° C. to minimize the gelatinization and then 105° C. overnight to obtain the dry weight.

Tables 7 and 8 below show the product yields (percent of dry solids of each fraction per 100 g dry matter of corn) for control and enzymatic runs in both experiments.

TABLE 7
Fraction yields for the conventional and
enzymatic samples in Experiment 1.

| | Steep | | |
|---|---|---|---|
| | 1A | 1B | 1C |
| | Conven- | 125 μg | 125 μg |
| | tional | Cellulase F | Cellulase G |
| starch + Gluten | 75.61% | 77.37% | 76.68% |
| Fiber | 9.35% | 8.67% | 8.64% |
| Germ | 6.46% | 5.56% | 5.63% |
| LSW Solubles | 4.97% | 4.00% | 3.90% |
| Filtrate Solubles | 2.12% | 3.61% | 2.41% |

TABLE 8
Fraction yields for the conventional and
enzymatic samples in Experiment 2.

| | Steep | | | | |
|---|---|---|---|---|---|
| | | 2B | 2C | 2D | 2E |
| | 2A | 50 μg | 50 μg | 25 μg | 25 μg |
| | Conven- | Cellu- | Celluase | Cellu- | Cellu- |
| | tional | lase F | G | lase F | lase G |
| starch + gluten | 74.15% | 75.98% | 75.63% | 75.58% | 75.15% |
| Fiber | 10.08% | 9.43% | 9.71% | 9.75% | 10.23% |
| Germ | 5.93% | 5.70% | 5.45% | 5.66% | 5.58% |
| LSW Solubles | 4.83% | 3.56% | 3.69% | 3.63% | 3.72% |
| Filtrate Solubles | 1.60% | 2.15% | 2.02% | 2.70% | 2.85% |

The starch plus gluten data from these two experiments in Table 9 below showed that Cellulase F, including a beta-xylosidase component, outperformed Cellulase G, including xylanse and cellulase components only, and which does not include a beta-xylosidase component, at different dosages.

TABLE 9
Starch&gluten yields for the enzymatic sample in Experiment 1 & 2

| | Steep | | | | | |
|---|---|---|---|---|---|---|
| | 1B | 1C | 2B | 2C | 2D | 2E |
| Enzyme | 125 μg | 125 μg | 50 μg | 50 μg | 25 μg | 25 μg |
| | Cellulase F | Cellulase G | Cellulase F | Cellulase G | Cellulase F | Cellulase G |
| Starch plus gluten | 77.37% | 76.68% | 75.98% | 75.63% | 75.58% | 75.15% |

### Example 3

### Wet Milling with Cellulase F, Cellulase G and Proteases

Two experiments (designated Experiment 3 and Experiment 4) were conducted to compare the performance of Cellulase F, which includes a beta-xylosidase component, and Cellulase G including xylanse and cellulase components only, and which does not include a beta-xylosidase component, blending with proteases in which three corn steeps were assembled and ground, respectively, to simulate the industrial corn wet milling process. They were processed individually using the same equipment and methodology. Each experiment included three enzymatic steps (Experiment 3, steep 3A, 3B, 3C and 3D; Experiment 4, steep 4A, 4B, 4C and 4D). The various process steps are described below.

The moisture of the corn used in the experiment was determined by loss in weight during oven drying. The corn that was used was weighed and placed in a 105° C. oven for 72 hours. The corn was then re-weighed after oven drying. The loss in weight was used to determine the corn's original solids content.

Steeping: The enzymatic sample (steep A to D) was steeped in a 0.06% (w/v) SO2 and 0.5% (w/v) lactic acid solution for 16 hours prior to milling. 100 grams of dry corn was put into 200 mL of the steep water described above. The entire mixture was then put into an orbital air heated shaker machine which was set to 175 RPM at 52° C. and allowed to mix at this temperature for desired time. At the end of the steeping process, the corn mixture was poured over a Buchner funnel for dewatering, and 100 mL of fresh tap water was added to the original steeping flask for rinsing purposes. It was then poured over the corn as a wash and captured in the same flask as the original corn draining. The purpose of this washing step was to retain as many of the solubles with the filtrate as possible. The total light steepwater fraction was placed into a tared flask and oven-dried completely at 105° C. for 24 hours. The flask was weighed post-drying to determine the amount of dry substance present.

First Grind: The corn was then placed into a Waring Laboratory Blender with the blades reversed (so the leading edge was dull). 200 mL of water was added to the corn in the blender, along with the corn rinsewater from above, and the corn was ground for one minute to facilitate germ release. 50 mL of fresh water was used to rinse out the blender and was then poured into the plastic bucket along with the first grind material. Then the slurry was transferred back to each flask and enzymes (labeled A to D, 3A and 4A was relevant control) were added as the ratio shown below in Table 10. The flask with corn slurry was transferred to orbital shaker and incubated at 52° C. for 4 hours. After incubation, the slurry was poured out to a 5 L plastic bucket for manual germ removal.

TABLE 10
Experimental design

| | Cellulase (25 μg | Protease (2.5 μg |
|---|---|---|
| Steeps | EP/g dry corn) | EP/g dry corn) |
| 3A | Cellulase F | Not added |
| 3B | | Protease C |
| 3C | | Protease B |
| 3D | | Protease 1 |
| 4A | Cellulase G | Not added |
| 4B | | Protease C |
| 4C | | Protease B |
| 4D | | Protease 1 |

De-germination: A slotted spoon was used to gently stir the mixture briefly. After the stirring was stopped, large quantities of germ pieces floated to the surface. These were skimmed off of the liquid surface using the slotted spoon.

The germ pieces were placed on a US No. 100 screen with a catch pan underneath of it. This process of mixing and skimming was repeated until negligible germ floated up to the surface for skimming. Inspection of the settled slurry mash in the slotted spoon also showed no evidence of large germ quantities left in the mixture at this point, so de-germination was stopped.

The germ pieces that had been accumulated on the No. 100 screen were transferred to a small beaker and swirled around with 125 mL of fresh tapwater to wash as much of the starch off of the germ as possible.

The germ and water in the beaker were poured back over the 100 mesh screen for dewatering. The degerminated slurry in the bucket was then poured back into the blender for a second grind. The water that passed through the 100 mesh screen from the 1st germ wash was then used to rinse the plastic bucket into the blender. A second 125 mL aliquot of tapwater was then poured over the germ pieces on the screen to facilitate further washing. This water was collected again in the catch pan and used as a second rinse of the plastic bucket into the blender. The germ on the screen was then pressed with a spatula and transferred to a tared weight pan and oven dried for 24 hours at 105° C. before analysis.

Second grind: The fiber, starch, and gluten slurry that had been de-germinated was then ground in the blender for 3 minutes with the high speed. This increased speed was employed to release as much starch and gluten from the fiber as possible.

Fiber Washing: With the second grind complete, the slurry in the blender was screened over a No. 100 vibrating screen (Retsch Model A200 sieve shaking unit). The shaking frequency on the Retsch unit was set to roughly 60 HZ. Once filtration had stopped, the starch and gluten filtrate portion was transferred into a flask for storage until tabling. 500 mL of fresh water was then used to rinse the blender after the second grind into a plastic bucket. The fiber on the top of the fiber screen was then added to the plastic bucket, swirled around in the 500 mL of fresh water and then re-screened. The filtrate from this washing was then transferred to the storage flask along with the first batch of filtrate.

The fiber was then washed and screened in this manner three successive times, each time using 240 mL of fresh wash water. This was then followed by a single 125 mL wash while vibrating to achieve maximum starch and gluten liberation from the fiber fraction. After all washings were complete, the fiber was gently pressed on the screen to dewater it before it was transferred to a tared aluminum weighing pan for oven drying at 105° C. for 24 hours prior to weighing.

All of the filtrate from the washings and pressing was added to the storage flask, yielding a total starch and gluten slurry volume of approximately 1,800 mL.

The starch and gluten slurry was then vacuum filtered through a Buchner Funnel through a Whatman filter paper before being oven dried. The total filtrate volume from the vacuum flask was measured. 250 ml filtrate was transferred to a plastic bottle for oven drying at 105° C. for 48 hours. The total soluble solid content of this fraction was calculated by multiplying the volume of gluten solution by total solids of gluten filtrate. The filter cake was transferred to a stainless steel dish to dry overnight fist at 50° C. to minimize the gelatinization and then 105° C. overnight to obtain the dry weight.

Tables 11 and 12 below show the product yields (percent of dry solids of each fraction per 100 g dry matter of corn) for control and enzymatic runs in both experiments.

TABLE 11
Fraction yields for the conventional and
enzymatic samples in Experiment 3.

| | Steeps | | | |
|---|---|---|---|---|
| | 3A | 3B | 3C | 3D |
| | Cellu- | Cellulase F & | Cellulase F & | Cellulase F & |
| | lase F | Protease C | Protease B | Protease 1 |
| starch + | 72.60% | 73.68% | 74.11% | 74.67% |
| gluten | | | | |
| Fiber | 11.08% | 10.89% | 10.34% | 9.78% |
| Germ | 5.65% | 5.40% | 5.33% | 5.46% |
| LSW | 3.53% | 3.41% | 3.34% | 3.38% |
| Solubles | | | | |
| Filtrate | 2.06% | 3.03% | 2.79% | 3.37% |
| Solubles | | | | |

TABLE 12
Fraction yields for the conventional and
enzymatic samples in Experiment 4.

| | Steeps | | | |
|---|---|---|---|---|
| | 4A | 4B | 4C | 4D |
| | Cellu- | Cellulase G & | Cellulase G & | Cellulase G & |
| | lase G | Protease C | Protease B | Protease 1 |
| Starch | 74.92% | 74.73% | 75.50% | 75.74% |
| Fiber | 10.52% | 10.48% | 9.82% | 9.51% |
| Germ | 5.59% | 5.56% | 5.47% | 5.49% |
| LSW | 3.72% | 3.66% | 3.60% | 3.60% |
| Solubles | | | | |
| Filtrate | 2.70% | 2.78% | 2.59% | 3.15% |
| Solubles | | | | |

The starch+gluten yield of two experiments was divided with the relevant control (3A,4A) to compare the boosting effect of different proteases to Cellulase F or Cellulase G (where control is Cellulase F alone or Cellulase G alone, respectively). The results in Table 13 showed that Cellulase F, which includes a beta-xylosidase component, blends with proteases could achieve higher starch+gluten yield compared with Cellulase G, including xylanse and cellulase components only, and which does not include a beta-xylosidase component, blends with proteases.

TABLE 13
Relative Starch + Gluten yields (%) to control for the
enzymatic sample in Experiment 3 & 4

| | Steeps | | | | | |
|---|---|---|---|---|---|---|
| | 3B | 3C | 3D | 4B | 4C | 4D |
| | Cellulase F | Cellulase F | Cellulase F | Cellulase | Cellulase | Cellulase G & |
| | & Protease C | & Protease B | & Protease 1 | G & | G & | Protease 1 |
| | | | | Protease C | Protease B | |
| Relative | 101.49% | 102.07% | 102.86% | 99.74% | 100.78% | 101.09% |
| starch + gluten | | | | | | |
| yield | | | | | | |

### Example 4

### Wet Milling with Beta-Xylosidase and Endoglucanase

An experiment was conducted to evaluate whether Aspergillus fumigatus beta-xylosidase (WO 2011/057140) could boost T. reesei endoglucanase EGI (Cellulase I) in corn wet milling process in which 5 corn steeps were assembled and ground, respectively, to simulate the industrial corn wet milling process. They were processed individually using the same equipment and methodology. The experiment included one the conventional steep (steep A) and the rest were enzymatic steps (steep B, C, D and E). The various process steps are described below.

The moisture of the corn used in the experiment was determined by loss in weight during oven drying. The corn that was used was weighed and placed in a 105° C. oven for 72 hours. The corn was then re-weighed after oven drying. The loss in weight was used to determine the corn's original solids content.

Steeping: The conventional sample (steep A) was steeped in a 0.15% (w/v) SO2 and a 0.5% (w/v) lactic acid solution for 28 hours prior to milling. The enzymatic sample (steep B to E) was steeped in a 0.06% (w/v) SO2 and 0.5% (w/v) lactic acid solution for 16 hours prior to milling. 100 grams of dry corn was put into 200 mL of the steep water described above. The entire mixture was then put into an orbital air heated shaker machine which was set to 175 RPM at 52° C. and allowed to mix at this temperature for desired time. At the end of the steeping process, the corn mixture was poured over a Buchner funnel for dewatering, and 100 mL of fresh tap water was added to the original steeping flask for rinsing purposes. It was then poured over the corn as a wash and captured in the same flask as the original corn draining. The purpose of this washing step was to retain as many of the solubles with the filtrate as possible. The total light steepwater fraction was placed into a tared flask and oven-dried completely at 105° C. for 24 hours. The flask was weighed post-drying to determine the amount of dry substance present.

First Grind: The corn was then placed into a Waring Laboratory Blender with the blades reversed (so the leading edge was dull). 200 mL of water was added to the corn in the blender, along with the corn rinsewater from above, and the corn was ground for one minute to facilitate germ release. 50 mL of fresh water was used to rinse out the blender and was then poured into the plastic bucket along with the first grind material. Then the slurry was transferred back to each flask and enzymes(Steep B to E) were added as shown below in table 14. The flask with corn slurry was transferred to orbital shaker and incubated at 52° C. for 4 hours. After incubation, the slurry was poured out to a 5 L plastic bucket for manual germ removal.

TABLE 14
Experimental Design

| | Steeps | | | | |
|---|---|---|---|---|---|
| | A | B | C | D | E |
| | Enzyme Used | | | | |
| | | 100% | 75% | 100% | 75% |
| | Conven- | Endoglu- | Endoglu- | Endoglu- | Endoglu- |
| | tional | canase | canse | canase | canase |
| | No- | 0% beta- | 25% beta- | 0% beta- | 0% beta- |
| | Enzyme | xylosidase | xylosidase | xylosidase | xylosidase |
| μg EG | 0 | 50 | 37.5 | 250 | 187.5 |
| μg BX | 0 | 0 | 12.5 | 0 | 62.5 |
| Total μg | 0 | 50 | 50 | 250 | 250 |
| EP/g | | | | | |
| dry corn | | | | | |

The conventional (no enzyme) treatment was ground and treated as described below immediately after 28 hours steeping without 2^{nd} time incubation.

De-germination: A slotted spoon was used to gently stir the mixture briefly. After the stirring was stopped, large quantities of germ pieces floated to the surface. These were skimmed off of the liquid surface using the slotted spoon.

The germ pieces were placed on a US No. 100 screen with a catch pan underneath of it. This process of mixing and skimming was repeated until negligible germ floated up to the surface for skimming. Inspection of the settled slurry mash in the slotted spoon also showed no evidence of large germ quantities left in the mixture at this point, so de-germination was stopped.

The germ pieces that had been accumulated on the No. 100 screen were transferred to a small beaker and swirled around with 125 mL of fresh tapwater to wash as much of the starch off of the germ as possible.

The germ and water in the beaker were poured back over the 100 mesh screen for dewatering. The degerminated slurry in the bucket was then poured back into the blender for a second grind. The water that passed through the 100 mesh screen from the 1st germ wash was then used to rinse the plastic bucket into the blender. A second 125 mL aliquot of tapwater was then poured over the germ pieces on the screen to facilitate further washing. This water was collected again in the catch pan and used as a second rinse of the plastic bucket into the blender. The germ on the screen was then pressed with a spatula and transferred to a tared weight pan and oven dried for 24 hours at 105° C. before analysis.

Second grind: The fiber, starch, and gluten slurry that had been de-germinated was then ground in the blender for 3 minutes with the high speed. This increased speed was employed to release as much starch and gluten from the fiber as possible.

Fiber Washing: With the second grind complete, the slurry in the blender was screened over a No. 100 vibrating screen (Retsch Model A200 sieve shaking unit). The shaking frequency on the Retsch unit was set to roughly 60 HZ. Once filtration had stopped, the starch and gluten filtrate portion was transferred into a flask for storage until tabling. 500 mL of fresh water was then used to rinse the blender after the second grind into a plastic bucket. The fiber on the top of the fiber screen was then added to the plastic bucket, swirled around in the 500 mL of fresh water and then re-screened. The filtrate from this washing was then transferred to the storage flask along with the first batch of filtrate.

The fiber was then washed and screened in this manner three successive times, each time using 240 mL of fresh wash water. This was then followed by a single 125 mL wash while vibrating to achieve maximum starch and gluten liberation from the fiber fraction. After all washings were complete, the fiber was gently pressed on the screen to dewater it before it was transferred to a tared aluminum weighing pan for oven drying at 105° C. for 24 hours prior to weighing.

All of the filtrate from the washings and pressing was added to the storage flask, yielding a total starch and gluten slurry volume of approximately 1,800 mL.

The starch and gluten slurry was then vacuum filtered through a Buchner Funnel through a Whatman filter paper before being oven dried. The total filtrate volume from the vacuum flask was measured. 250 ml filtrate was transferred to a plastic bottle for oven drying at 105° C. for 48 hours. The total soluble solid content of this fraction was calculated by multiplying the volume of gluten solution by total solids of gluten filtrate. The filter cake was transferred to a stainless steel dish to dry overnight fist at 50° C. to minimize the gelatinization and then 105° C. overnight to obtain the dry weight.

Table 15 below shows the product yields (percent of dry solids of each fraction per 100 g dry matter of corn) for control and enzymatic runs in both experiments.

TABLE 15
Fraction yields for the conventional and enzymatic samples

| | Steep | | | | |
|---|---|---|---|---|---|
| | A | B | C | D | E |
| | Conven- | Low | Low | High | High |
| | tional | dose | dose | dose | dose |
| | No- | 100% EG | 75% EG | 100% EG | 75% EG |
| | enzyme | 0% BX | 25% BX | 0% BX | 25% BX |
| starch + | 76.35% | 75.66% | 76.06% | 76.62% | 76.64% |
| Gluten | | | | | |
| Fiber | 9.46% | 10.35% | 9.93% | 8.82% | 9.06% |
| Germ | 5.40% | 5.32% | 5.37% | 5.33% | 5.35% |
| LSW | 4.93% | 3.61% | 3.62% | 3.60% | 3.54% |
| Solubles | | | | | |
| Filtrate | 1.61% | 2.08% | 1.80% | 2.95% | 2.92% |
| Solubles | | | | | |

Table 15 shows that the addition of beta-xylosidase(BX) to endo-glucanase(EG) at 50 ug/g dry corn resulted in higher starch and gluten yields than endoglucanase alone.

1. A process for treating crop kernels, comprising the steps of:
a) soaking kernels in water to produce soaked kernels;
b) grinding the soaked kernels; and
c) treating the soaked kernels in the presence of an effective amount of a beta-xylosidase;
1. wherein step c) is performed before, during or after step b).

2. The process of claim 1, further comprising treating the soaked kernels in the presence of a protease.

3. The process of claim 1, further comprising treating the soaked kernels in the presence of a cellulolytic composition.

4. The process of claim 1, further comprising treating the soaked kernels in the presence of an enzyme selected from the group consisting of an endoglucanase, a xylanase, a cellobiohydrolase I, a cellobiohydrolase II, a GH61 polypeptide, or a combination thereof.

5. The process of claim 1, further comprising treating the soaked kernels in the presence of an endoglucanase.

6. The process of claim 1, further comprising treating the soaked kernels in the presence of a xylanase.

7. The process of claim 1, wherein the kernels are soaked in water for about 2-10 hours, preferably about 3 hours.

8. The process of claim 1, wherein the soaking is carried out at a temperature between about 40° C. and about 60° C.

9. The process of claim 1, wherein the soaking is carried out at acidic pH.

10. The process of claim 1, wherein the soaking is performed in the presence of between 0.01-1% SO2 and/or NaHSO3.

11. The process of claim 1, wherein the crop kernels are from corn (maize), rice, barley, sorghum bean, or fruit hulls, or wheat.

12. The process of claim 1, wherein the beta-xylosidase is derived from the genus Aspergillus.

13. The process of claim 1, wherein the beta-xylosidase has at least 80% identity to SEQ ID NO: 6.

14. The process of claim 1, further comprising treating the kernels with pentosanase, pectinase, arabinanase, arabinofurasidase, xyloglucanase, protease, and/or phytase.

15. (canceled)

16. The process of claim 1, wherein the beta-xylosidase enhances the wet milling benefit of one or more enzymes.


SEQUENCE LISTING (6 sequences)

SEQ ID NO: 1 - protein, 250 aa, Thermoascus aurantiacus
MSFSKIIATAGVLASASLVAGHGFVQNIVIDGKKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 2 - protein, 253 aa, Penicillium emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 3 - protein, 418 aa, Trichoderma reesei
MNKSVAPLLLAASILYGGAVAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNEDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 4 - protein, 296 aa, Aspergillus aculeatus
MARLSQFLLACALAVKAFAAPAAEPVEERGPNFFSALAGRSTGSSTGYSNGYYYSFWTDGASGDVEYSNGAGGSYSVTWSSASNFVGGKGWNPGSAHDITYSGSWTSTGNSNSYLSVYGWTTGPLVEYYILEDYGEYNPGSAGTYKGSVYSDGSTYNIYTATRTNAPSIQGTATFTQYWSIRQTKRVGGTVTTANHFNAWAKLGMNLGTHNYQIVATEGYYSSGSASITVAERADILLRYMLYLWHRFCDGNEWMRKLACSYMSRVVVSEFGALDFELFFIQSQPPVSQQVKKKKK

SEQ ID NO: 5 - protein, 397 aa, Aspergillus fumigatus
MVHLSSLAAALAALPLVYGAGLNTAAKAKGLKYFGSATDNPELTDSAYVAQLSNTDDFGQITPGNSMKWDATEPSQNSFSFANGDAVVNLANKNGQLMRCHTLVWHSQLPNWVSSGSWTNATLLAAMKNHITNVVTHYKGKCYAWDVVNEALNEDGTFRNSVFYQIIGPAYIPIAFATAAAADPDVKLYYNDYNIEYSGAKATAAQNIVKMIKAYGAKIDGVGLQAHFIVGSTPSQSDLTTVLKGYTALGVEVAYTELDIRMQLPSTAAKLAQQSTDFQGVAAACVSTTGCVGVTIWDWTDKYSWVPSVFQGYGAPLPWDENYVKKPAYDGLMAGLGASGSGTTTTTTTTSTTTGGTDPTGVAQKWGQCGGIGWTGPTTCVSGTTCQKLNDWYSQCL

SEQ ID NO: 6 - protein, 792 aa, Aspergillus fumigatus
MAVAKSIAAVLVALLPGALAQANTSYVDYNVEANPDLTPQSVATIDLSFPDCENGPLSKTLVCDTSARPHDRAAALVSMFTFEELVNNTGNTSPGVPRLGLPPYQVWSEALHGLDRANFTNEGEYSWATSFPMPILTMSALNRTLINQIATIIATQGRAFNNVGRYGLDVYAPNINAFRSAMWGRGQETPGEDAYCLASAYAYEYITGIQGGVDPEHLKLVATAKHYAGYDLENWDGHSRLGNDMNITQQELSEYYTPQFLVAARDAKVHSVMCSYNAVNGVPSCANSFFLQTLLRDTFGFVEDGYVSSDCDSAYNVWNPHEFAANITGAAADSIRAGTDIDCGTTYQYYFGEAFDEQEVTRAEIERGVIRLYSNLVRLGYFDGNGSVYRDLTWNDVVTTDAWNISYEAAVEGIVLLKNDGTLPLAKSVRSVALIGPWMNVTTQLQGNYFGPAPYLISPLNAFQNSDFDVNYAFGTNISSHSTDGFSEALSAAKKSDVIIFAGGIDNTLEAEAMDRMNITWPGNQLQLIDQLSQLGKPLIVLQMGGGQVDSSSLKSNKNVNSLIWGGYPGQSGGQALLDIITGKRAPAGRLVVTQYPAEYATQFPATDMSLRPHGNNPGQTYMWYTGTPVYEFGHGLFYTTFHASLPGTGKDKTSFNIQDLLTQPHPGFANVEQMPLLNFTVTITNTGKVASDYTAMLFANTTAGPAPYPNKWLVGFDRLASLEPHRSQTMTIPVTIDSVARTDEAGNRVLYPGKYELALNNERSVVLQFVLTGREAVIFKWPVEQQQISSA